(19) United States
(12) Patent Application Publication
(10) Pub. No.: US 2026/0102371 A1
(43) Pub. Date: Apr. 16, 2026

(54) PROCESS FOR SYNTHESIZING A FORMULATION OF GALANGIN-LOADED HYDROGEL FOR EXPEDITED WOUND HEALING

(71) Applicants: Khadijah B. ALKINANI, Makkah (SA); Abeer S. ALGHAMDI, Jeddah (SA); Ashraf B. ABDEL-NAIM, Jeddah (SA); Fahad A. ALABASSI, Jeddah (SA); Imran KAZMI, Jeddah (SA); Riya MISHRA, Jaipur (IN)

(72) Inventors: Khadijah B. ALKINANI, Makkah (SA); Abeer S. ALGHAMDI, Jeddah (SA); Ashraf B. ABDEL-NAIM, Jeddah (SA); Fahad A. ALABASSI, Jeddah (SA); Imran KAZMI, Jeddah (SA); Riya MISHRA, Jaipur (IN)

(21) Appl. No.: 19/412,201

(22) Filed: Dec. 8, 2025

(51) Int. Cl. A61K 31/352; A61K 9/06; A61K 47/38; A61P 17/02

(52) U.S. Cl. CPC A61K 31/352; A61K 9/06; A61K 47/38; A61P 17/02

(57) ABSTRACT

The present invention relates to a process for synthesizing a formulation of Galangin (GAL)-loaded hydrogel for Expedite wound healing. The formulation is prepared by mixing 5 gm GAL and 1.5 g of HPMC, wherein at room temperature, HPMC was dispersed in 100 ml of distilled water with stirring by a hotplate stirrer (LMS-1003), and the previously weighed GAL was gradually dissolved in the mixture with continuous stirring for 10 minutes. Beakers were then sealed with parafilm, stored at 4° C. for two days prior to utilization. This invention investigated the potential effects of galangin (GAL), on wound healing in streptozotocin-induced diabetic rats. The findings demonstrate that GAL is a promising topical agent for promoting diabetic wound healing through antioxidant, anti-inflammatory, pro-angiogenic, and pro-collagen activities.

### FIELD OF THE INVENTION

The present disclosure relates to the wound healing application, particularly to a process for synthesizing a formulation of Galangin (GAL)-loaded hydrogel for expedite wound healing. In the present invention, a Galangin-loaded hydrogel formulation is prepared and its efficacy is tested for wound healing, in streptozotocin-induced diabetic rats.

### BACKGROUND OF THE INVENTION

Diabetes is a global health concern affecting millions worldwide, with 23.1% of Saudi Arabia's population impacted by this chronic metabolic disorder characterized by elevated blood sugar levels stemming from insufficient insulin production or impaired utilization. This condition encompasses various types, including Type-I and Type-II diabetes mellitus, each posing distinct challenges and requiring tailored management approaches. A critical issue that arises is impaired wound healing, particularly diabetic foot ulcers (DFUs), referred to as persistent, non-healing, and damaged wounds, which affect up to 25% of diabetic patients worldwide. The delayed wound healing is caused by disruption in each phase of wound healing; hemostasis, inflammation, proliferation, and remodeling phases, which is attributed to oxidative stress, inflammation, reduced quantity of collagen, neuropathy, and compromised vascularization, heightening the risk of chronic ulcers, infections, or gangrene, with over 84% of cases culminating in lower limb amputation.

The intricate relationship between diabetes and wound healing involves intricate cellular and molecular mechanisms. Elevated blood sugar levels can impair the function of crucial cells involved in wound repair, such as fibroblasts and macrophages. Additionally, neuropathy and vascular complications further contribute to the challenge by diminishing sensation and reducing blood flow to affected areas. Consequently, chronic wounds, infections, and ulcers become more prevalent among individuals with diabetes, necessitating a comprehensive approach that addresses both the underlying condition and the specific needs of wound care in this population.

Galangin (GAL), a naturally occurring flavonoid derived from the root of Alpinia officinarum and honey, possesses recognized anti-inflammatory and angiogenic properties. Various literates showed that neuro-protective effects of the GAL and could be beneficial in the management of various conditions, including neurodegenerative diseases. Moreover, GAL has exhibited antimicrobial properties against bacteria and viruses, along with the potential to modulate immune responses. All these observations indicate the potential of GAL as a solution for addressing the aforementioned problem of wound healing in case of diabetes.

In the view of the foregoing discussion, the present invention provides a Galangin-loaded hydrogel formulation is prepared and its efficacy is tested for wound healing, in streptozotocin-induced diabetic rats.

### SUMMARY OF THE INVENTION

The present disclosure relates to a process for synthesizing a formulation of Galangin (GAL)-loaded hydrogel for expedite wound healing. The present disclosure aims to evaluate the potential effects of galangin (GAL), a natural flavonoid from Alpinia officinarum, on wound healing in streptozotocin-induced diabetic rats. Fifty male Wistar rats were allocated into five groups (n=10): normal control, untreated diabetic, vehicle-treated diabetic, positive control, and GAL-treated diabetic. This was followed by the creation of a standardized 1-cm circular wound on the dorsal skin. Treatments were applied topically for 14 consecutive days, and wounds were photographed and measured on days 0, 3, 7, 10, and 14. The GAL-treated group showed a significantly higher rate of wound contraction. Histopathological analysis revealed the absence of scab, full re-epithelialization, neovascularization. GAL treatment significantly improved oxidative status, as evidenced by decreased MDA, increased SOD and CAT, and elevated expression of Nrf2 and NQO1. GAL also effectively modulated inflammatory responses by suppressing key pro-inflammatory mediators (IL-6, TNF-α, IL-13, COX-2, and TLR4) were suppressed. Additionally, GAL enhanced angiogenesis via VEGF-A, PDGFR, and TGF-01, and promoted collagen deposition through upregulation of Col1A1 and Col4A1. Collectively, the findings demonstrate that GAL is a promising topical agent for promoting diabetic wound healing through antioxidant, anti-inflammatory, pro-angiogenic, and pro-collagen activities.

An object of the present disclosure is to provide a process for synthesizing a formulation of Galangin (GAL)-loaded hydrogel for expedite wound healing.

Another object of the present disclosure is to investigate the potential effects of galangin (GAL) on wound healing in streptozotocin-induced diabetic rats.

Another object of the present disclosure is to apply GAL treatment topically to address impaired wound healing in diabetes mellitus.

Another object of the present disclosure is to address chronic hyperglycemia that contributes to excessive oxidative stress, persistent inflammation, and impaired angiogenesis-factors that collectively hinder the healing process.

Another object of the present disclosure is to provide a promising topical agent for promoting diabetic wound healing through antioxidant, anti-inflammatory, pro-angiogenic, and pro-collagen activities.

Yet, another object of the present disclosure is to reduce the considerable socioeconomic burden imposed by chronic ulcers that frequently lead to lower extremity amputations.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a standard calibration curve of Malondialdehyde (MDA), in accordance with an embodiment of the present disclosure;

FIG. 2 illustrates a table showing the RT master mix (per 20-μL reaction), in accordance with an embodiment of the present disclosure;

FIG. 3 illustrates a table showing the primer sequence, in accordance with an embodiment of the present disclosure;

FIG. 4A illustrates the effect of GAL on the wound healing, in accordance with an embodiment of the present disclosure;

FIG. 4B illustrates a graphical representation of the effect of GAL on wound contraction in diabetic rats, in accordance with an embodiment of the present disclosure;

FIG. 5 illustrates a table showing Histological features of wound healing in rats treated with GAL on day 14, in accordance with an embodiment of the present disclosure;

FIG. 6 illustrates Sections of wounded rat skin tissues stained with both H&E and MTC to compare the healing processes, in accordance with an embodiment of the present disclosure;

FIG. 7A illustrates a graphical representation of MDA concentration, as effects of GAL on oxidative stress and antioxidant markers, in accordance with an embodiment of the present disclosure;

FIG. 7B illustrates a graphical representation of SOD activity, as effects of GAL on oxidative stress and antioxidant markers, in accordance with an embodiment of the present disclosure;

FIG. 7C illustrates a graphical representation of CAT activity, as effects of GAL on oxidative stress and antioxidant markers, in accordance with an embodiment of the present disclosure;

FIG. 8 illustrates an assessment of immunoreactivity expression of NQO1 and Nrf2 in diabetic rat skin tissues, in accordance with an embodiment of the present disclosure;

FIG. 9 illustrates Immunohistochemical analysis with quantitative assessment of immunopositive reaction of inflammatory markers expression in diabetic rat skin tissues, in accordance with an embodiment of the present disclosure;

FIG. 10 illustrates Immunohistochemical analysis with quantitative assessment of immunopositive reaction of angiogenic markers expression in diabetic rat skin tissues, in accordance with an embodiment of the present disclosure;

FIG. 11A illustrates assessment mRNA expression of Col1A1, showing effects of GAL on mRNA fold change expression of collagen genes, in accordance with an embodiment of the present disclosure; and

FIG. 11B illustrates assessment mRNA expression of Col4A1, showing effects of GAL on mRNA fold change expression of collagen genes, in accordance with an embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

The present invention relates to a process for synthesizing a formulation of Galangin (GAL)-loaded hydrogel for expedite wound healing.

In an embodiment, the invention aims to use several drugs and chemical including: GAL; Streptozotocin; MEBO; Ketamine; Xylazine; Hydroxypropyl methyl cellulose; Tri-Sodium citrate (dihydrate); Citric acid (monohydrate); Hydrochloric Acid; Formaldehyde; and Dextrose. The following reagents were used: hydroxypropyl methyl cellulose (HPMC) for hydrogel preparation, diaminobenzidine: used for immunohistochemical staining, hematoxylin and Eosin (H and E) used for staining sections for histopathological examination. Masson's trichrome stain (MTC) used for staining sections for histopathological examination. Hydrogen peroxide solution: used for immunohistochemical staining, ethyl alcohol used for histopathological examination. Primary antibodies are as follows: Multiclonal anti-IL-1 beta (Cat #ab283818), Monoclonal anti-IL-6 (Cat #ab9324), Monoclonal anti-TNF-alpha (Cat #ab220210), Monoclonal anti-TGF beta 1 antibody (Cat #ab169771), Monoclonal anti-VEGFA antibody (Cat #ab1316), Monoclonal anti-PDGFR-alpha+PDGFR-beta antibody (Cat #ab32570), Monoclonal anti-COX-2 (Cat #ab52894), Monoclonal anti-Nrf2 (Cat #ab313825), Monoclonal anti-NQO1(Cat #ab97385), and Monoclonal anti-TLR4 (Cat #ab22048). Kits for oxidative stress markers were obtained, and includes colorimetric kits for assessment of MDA content (Cat #MD2529), CAT activity kit (Cat #CA2517) and SOD activity (Cat #SD252). Mouse and Rabbit HRP/DAB detection IHC kits (Cat #a64264)) were used for immunohistochemical assessment. RNA extraction kit (Cat #A27828, MagMAX mirVana and total RNA isolation kit) were used for real-time polymerase chain reaction (qPCR). cDNA Reverse Transcription Kit (Cat #4368814) was used for real-time polymerase chain reaction (qPCR). Taq PCR Master Mix kit (Cat #201445) was used for real-time polymerase chain reaction (qPCR). Male Wistar rats (190-230 g) were procured from the animal facility (Faculty of Pharmacy, King Abdulaziz University (KAU)). They were housed in standard laboratory environments (30-70% humidity, temperature (22±2° C.), and 12/12 h light-dark cycle).

For the preparation of the Galangin (GAL)-loaded hydrogel formulation, Five g of GAL and 1.5 g of HPMC were weighed by analytical balance (RADWAG AS 110/X). Then, at room temperature, HPMC was dispersed in 100 ml of distilled water with stirring by a hotplate stirrer (LMS-1003), and the previously weighed GAL was gradually dissolved in the mixture with continuous stirring for 10 minutes. Beakers were then sealed with parafilm, stored at 4° C. for two days prior to utilization.

To prepare the vehicle gel, 1.5 g of HPMC was dispersed in 100 ml of distilled water with magnetic stirring for 10 minutes. The hydrogels were then stored at 4° C. and allowed to swell for two days prior to conducting experiments.

Diabetes in rats was induced using a freshly prepared STZ solution in cold citrate buffer with a concentration of 0.1 M and a pH of 4.5. The solution was injected intraperitoneally (I.P.) at a dose of 50 mg/kg. The rats were given orally 5% dextrose for three days. After 7 days, blood glucose levels were measured, and only the rats with glucose levels ranging from 200 to 300 mg/dL were allocated into the experimental groups.

A total of 55 adult male Wistar rats were used in the experiment. After induction of diabetes and wounding, five rats died during the study due to anesthesia and diabetes-related complications, leaving 50 rats for final analysis. Rats were sorted at random into 5 groups and labelled with the appropriate numbers (n=10 for each group). These were as follows:

Group 1: Normal control group, untreated non-diabetic rats.

Group 2: Negative control group, untreated-diabetic rats.

Group 3: Rats treated with the drug-free hydrogel, vehicle-treated diabetic rats.

Group 4: Positive control group, treated with MEBO® ointment, Gulf Pharmaceutical Industries (with β-Sitosterol as the active ingredient, in a base of beeswax and sesame oil).

Group 5: Rats treated only with GAL-loaded hydrogel, GAL-treated diabetic rats.

All treatments were applied topically for a total of 14 days. Wound diameters (WDs) were measured, and photographs of the wounds were taken on days 0, 3, 7, 10, and 14. On day 15, all the rats were sacrificed by decapitation, and then the skin around the wound was dissected. One section of each skin was preserved in 10% neutral formalin, while the other section was immediately frozen in liquid nitrogen and kept at −80° C. for the biochemical assessments.

To bring the diabetic rats in anaesthetic state, ketamine and xylazine (100 mg/kg and 10 mg/kg body weight) was administered intraperitoneally. The shaved skin on the dorsal surface of their bodies was disinfected with ethanol (70%). After this, a 1×1 cm² area was removed from the dorsal surface of the rat, together with the epidermal, dermal, and subcutaneous layers of skin, as well as the connective tissue beneath the skin. Every diabetic rat was isolated in its own cage and provided with free access to water and food pellets.

The percentage of wound contraction was determined through the following formula:

$$Wound contraction \% = ( WD on day 0 - WD on day 14 ) / ( WD on day 0 ) \times 100$$

Immediately after sacrifice, skin tissues were excised out on ice, rinsed with a PBS solution, pH 7.4, to clean it from any red blood cells and, clots and then divided into two parts. One portion was kept in 10% neutral buffered formalin, and the rest was placed at −80° C.

The wounded skin was dissected out and stored in 10% formalin for 24 h, after that skin tissue was cut into small pieces and embedded in hot paraffin wax blocks. A microtome was used to cut a thin segment that measured 4 micrometers in thickness.

Following the meticulous rinsing of the collected skin specimens with cold saline, then carefully pressing the skin between two filter papers to dry the skin. The dried skins were then weighed and homogenized in phosphate-buffered saline (PBS) solution for obtaining 10% w/v tissue homogenate, followed by centrifugation of homogenate tissue at 3000 rpm for 20 minutes at 4 C°. In addition to measuring the superoxide dismutase (SOD), Catalase (CAT) activities as antioxidant indicators, the levels of malondialdehyde (MDA) were also assessed.

The skin tissues, kept in formalin, were washed in tap water then serial dilutions of alcohol (methyl, ethyl and absolute ethyl) and xylene were used for dehydration. Specimens were cleared in xylene and embedded in paraffin then formulated into paraffin blocks. After that, they were cut using a sledge tissue microtome into 4 μm thickness tissue sections. The tissue sections were then deparaffinized, rehydrated and stained with hematoxylin and eosin (H and E) and Masson's trichrome (MTC). The slides were examined by a pathologist, in a blind manner, with a Nikon light microscope to look for signs of possible structural damage.

For the detection of the degree of tissue damage, healing, formation of new tissue, collagen deposition, and fibroblast proliferation each section was scored from (− to +++).

Lipid peroxidation was evaluated by examining the levels of TBARS that were articulated as MDA levels. MDA is the result of the course of action of lipid peroxidation and acts as an indicator of the method. The test principle depends on the colorimetric determination of a pink color, ensuing from the reaction of TBARS with thiobarbituric acid (TBA) in an acidic medium (pH 2-3) after boiling.

The kit content includes: Thiobarbituric acid (TBA); Acetic acid to prepare the color reagent; Sodium hydroxide to prepare the color reagent; MDA Standard for preparing the standard curve; and Sodium dodecyl sulfate (SDS). An amount of 100 μl of standard or sample was taken in to a 5 ml vial. An amount of 100 μl of trichloroacetic Acid (TCA) solution was added to all vials and mixed. An amount of 4 ml of TBA was added downside of each vial. Vials were boiled for an hour. To stop the reaction, vials were instantly detached and positioned in ice bath for 10 min. Vials were revolved for 10 min at 1,600×g at 4 C°. An amount of 150 μl from the supernatants was encumbered to a clear plate. Absorbance was examined at 530 nm.

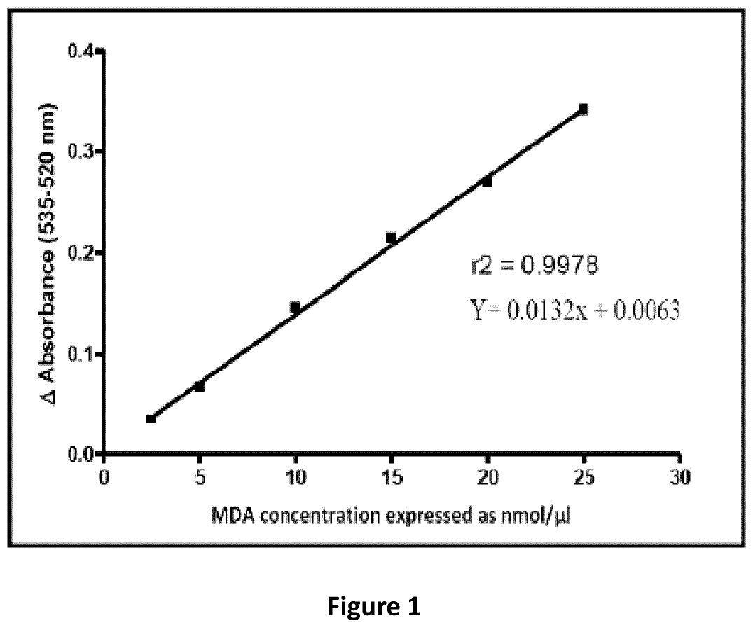

FIG. 1 illustrates a standard calibration curve of Malondialdehyde (MDA), in accordance with an embodiment of the present disclosure.

Referring to FIG. 1, the value of MDA for each sample were calculated from the standard curve.

The contents of TBARS were determined from the standard curve, multiplied by 10 and expressed as nmol/mg tissue.

In an embodiment, the test followed by utilizing tetrazolium salt to identify superoxide radicals released by hypoxanthine and xanthine oxidase. One SOD unit is described as the amount of enzyme dismutases 50% of superoxide radicals.

The kit content includes: assay buffer, sample buffer, radical detector that includes tetrazolium salt solution, SOD standard including bovine erythrocyte SOD (Cu/Zn), and Xanthine Oxidase.

For tissue preparation, skin tissues were rinsed by PBS, pH 7.4, and removed red blood cells and clots. Tissues were homogenized in HEPES (4-(2-hydroxyethyl)-1 piperazine ethane sulfonic acid) buffer, pH 7.2 contains ethylene glycol tetra acetic acid, sucrose, and mannitol.

An aliquot of 200 μl of the radical detector and 10 μl of the standard were added in the designated wells. Then, 200 μl of the radical detector and 10 μl of the samples were added in the sample wells. By adding 20 μl of diluted xanthine oxidase to every well the reaction was initiated. The plate was covered and incubated on a shaker for 30 min. The absorbance was study at 450 nm.

The activities of SOD were determined using the following equation

$$SOD ( U / ml ) = [ \text{} ( Sample LR - y - intercept / slope ) \times 0.23 ml / 0.01 ml ] \times sample dilution$$

Catalase (CAT) is involved in the detoxification of hydrogen peroxide (H₂O₂), a reactive oxygen species (ROS), which is a toxic product of both normal aerobic metabolism and pathogenic ROS production. This enzyme catalyzes the conversion of two molecules of H₂O₂ to molecular oxygen and two molecules of water (catalytic activity). CAT also demonstrates peroxidatic activity, in which low molecular weight alcohols can serve as electron donors. While aliphatic alcohols serve as specific substrates for CAT, other enzymes with peroxidatic activity do not utilize these substrates.

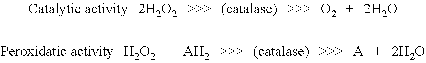

In humans, the highest levels of CAT are found in liver, kidney, and erythrocytes, where it is believed to account for the majority of H₂O₂ decomposition.

The kit content includes: CAT Assay Buffer; CAT Sample Buffer; CAT Formaldehyde Standard; CAT Control; CAT Potassium Hydroxide; CAT Hydrogen Peroxide; CAT Purpald (Chromogen); and CAT Potassium Periodate.

Formaldehyde Standard Wells—100 μl of Catalase Assay Buffer (1×), 30 μl of methanol, and 20 μl of standard were added per well in the designated wells on the plate. Then, Positive Control Wells—100 μl of Catalase Assay Buffer (1×), 30 μl of methanol, and 20 μl of diluted Catalase (Control) were added to two wells. Sample Wells—100 μl of Catalase Assay Buffer (1×), 30 μl of methanol, and 20 μl of sample were added to two wells. When necessary, samples were diluted with Catalase Sample Buffer (1×) or concentrated with an Amicon centrifuge concentrator with a molecular weight cut-off of 100,000. The reactions were initiated by adding 20 μl of diluted Hydrogen Peroxide to all the wells being used. The precise time the reaction was initiated was noted, and the diluted Hydrogen Peroxide was added as quickly as possible. The plate was covered with the plate cover and incubated on a shaker for 20 minutes at room temperature. 30 μl of Potassium Hydroxide was added to each well to terminate the reaction, and then 30 μl of Catalase Purpald (Chromogen) was added to each well. The plate was covered with the plate cover and incubated for 10 minutes at room temperature on the shaker. 10 μl of Catalase Potassium Periodate was added to each well. The plate was covered with the plate cover and incubated for five minutes at room temperature on a shaker. The absorbance was studied at 540 nm.

$$CAT activity = μM of sample / 20 min . \times sample dilution = nmol / min / ml$$

The protein expressions of IL-1β, IL-6, anti-TNF, TGF-1β, VEGFA, PDGER-alph+beta, COX-2, Nrf2, TLR4, and NQO1 were assessed using the immunohistochemical protein assessment technique.

Immunohistochemical protein detection and localization depends on the reaction between the target protein (Antigen) and specific antibody. Enzymes, such as Horseradish Peroxidase (HRP) is commonly used to catalyze a color-producing reaction. And the color can be detected by photographing using light microscope then quantified using image analysis software. Deparaffinized tissue sections were rehydrated with different ethanol concentration and boiled in citrate buffer (pH 6.0) for 10 minutes. After deparaffinization, sections were treated with 3% H₂O₂ in distilled H₂O for 10 min to quench the endogenous peroxide. A concentration of 10% goat serum was used to block nonspecific binding sites. The sections were then incubated for two hours in TBS containing 5% BSA, and then exposed overnight at 4° C. to the primary antibodies: Polyclonal anti-IL-6, Monoclonal anti-TNF, Monoclonal anti-TGF beta1 antibody, Monoclonal anti-VEGFA antibody and Monoclonal anti-PDGFR-beta, This was followed by incubation with biotinylated goat-anti-rabbit IgG, secondary antibodies, for 10 min. Sections were washed with PBS 3 times of 3 min each. This was followed by incubation for 10 min with streptavidin HRP. Antibody binding sites were detected by incubation with diaminobenzidine-H₂O₂ solution. Image analysis to calculate the optical density (OD) and area percent (A %) was performed using ImageJ software.

PCR is based on DNA polymerase enzyme-exponential copying of a part of a DNA molecule specifically increasing a target. Real time PCR detection methods depends on changes in fluorescence, which are proportional to the increase of the target. Wounded skin sections were homogenized by utilizing an ultra-probe sonicator. RNA extraction was performed using triazole solution, and an extraction kit of RNA.

Kit components include: Proteinase K, 50 mg/mL; Lysis/Binding Enhancer; TURBO DNase; Lysis Buffer; PK Digestion Buffer; RNA Binding Beads; Wash Solutions; Rebinding Buffer; DNase Buffer; Elution Buffer; Processing Plate; and Elution Plates.

Homogenized samples were mixed with magnetic beads. The beads were washed with wash buffers. RNA was then eluted off the beads. Then, a reverse transcription kit was utilized for the synthesis of cDNA.

The reagents include: RT buffer; RT random primers; dNTP mix (100 mM); MultiScribe™ Reverse Transcriptase, 50 U/μL; and RNase Inhibitor.

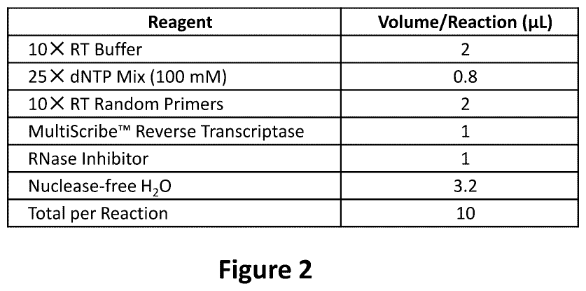

FIG. 2 illustrates a table showing the RT master mix (per 20-μL reaction), in accordance with an embodiment of the present disclosure.

The RT master mix (per 20-μL reaction) was prepared as shown in table shown in FIG. 2.

Ten microliters of 2×RT master mixture were pipetted into each individual tube. Ten microliters of RNA sample were pipetted into each well, pipetting up and down two times to mix. The tubes were sealed and briefly centrifuged to spin down the contents and eliminate any air bubbles. The thermal cycler conditions were programmed as following:

At step 1, the temperature is 25° C. and time is 10 min. At step 2, the temperature is 37° C., and time is 120 min. At step 3, the temperature includes 85° C., and time is 5 min. At step 4, the temperature is 4° C., and time is on hold. Reactions were loaded into the thermal cycler, and reverse transcription was started. For Amplification, the following kit components were used:
Taq DNA Polymerase
QIAGEN PCR Buffer
MgCl2
Ultrapure dNTPs

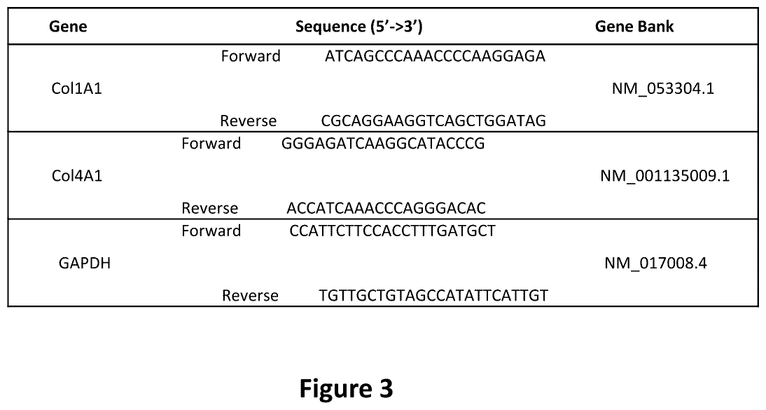

FIG. 3 illustrates a table showing the primer sequence, in accordance with an embodiment of the present disclosure.

A real-time PCR reaction mixture for each sample was prepared. The plate was sealed, mixed gently, then centrifuged briefly to spin down the contents and eliminate any air bubbles. The plate was run on an Applied Biosystems Step One real-time quantitative PCR instrument after adjusting the thermal cycle parameters. At the end, a melting curve for each primer was plotted and the RTPCR results were analyzed using the Applied Biosystems real-time PCR system software.

Gene expression changes were calculated by the comparative cycle threshold (Ct) method, and the values were normalized to endogenous reference GAPDH according to the following equations.

$$Δ {Ct}_{sample} = {Ct}_{gene of interest} - {Ct}_{housekeeping gene}$$

$$Δ Δ Ct = Δ {Ct}_{sample} - Δ C {t}_{control group}$$

$$RQ = 2 -- ΔΔ Ct$$

Wherein, Ct=cycle threshold, RQ=relative quantitation (indicates expression).

Data are shown as mean±standard deviation (SD). Unless otherwise stated, one-way analysis of variance (ANOVA) with Tukey's post hoc test was used to determine statistical significance. At a p-value <0.05, differences between samples were considered significant. The GraphPad Prism program, version 10.6.1 for Windows, was used for all analyses.

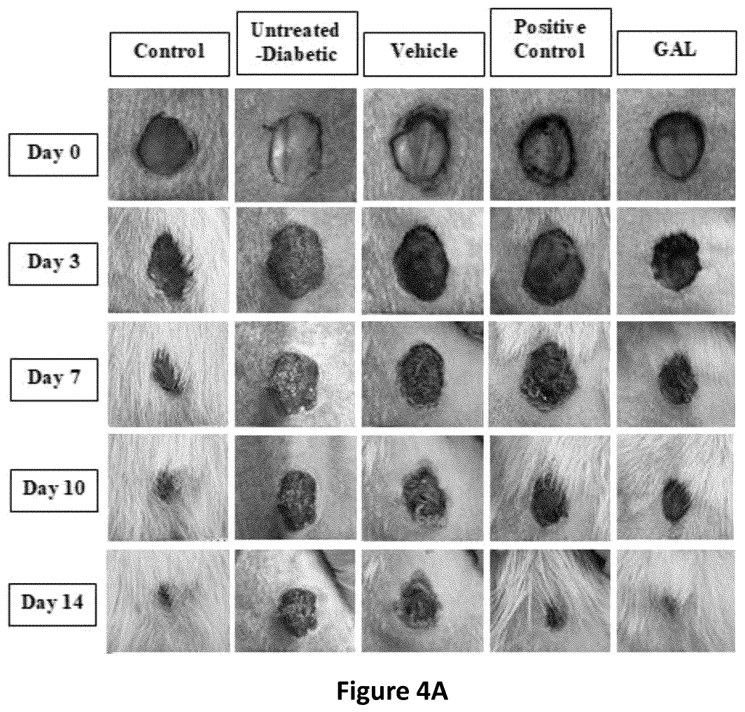

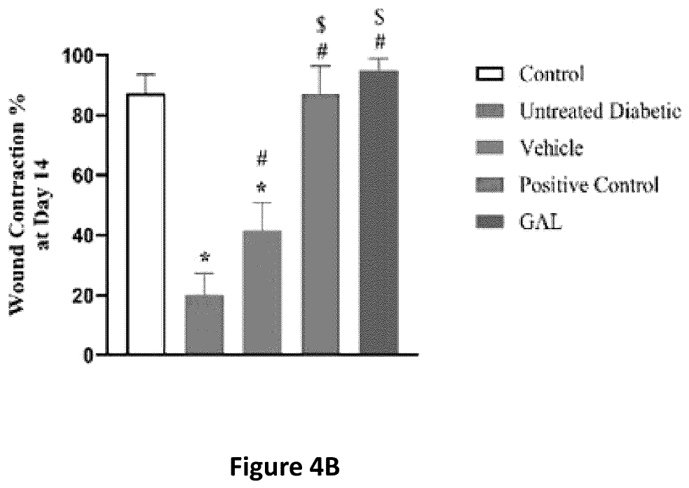

FIG. 4A illustrates the effect of GAL on the wound healing, in accordance with an embodiment of the present disclosure.

FIG. 4B illustrates a graphical representation of the effect of GAL on wound contraction in diabetic rats, in accordance with an embodiment of the present disclosure.

The impact of GAL on wound skin diameter, as an index of wound healing progression from day 0 to the 14^{th} day in different experimental diabetic rat groups was illustrated in (FIGS. 4A, and 4B). On day 0, all experimental groups were presented with open, full-thickness skin wounds of uniform dimensions. Topical administration of vehicle, positive control, and GAL significantly reduced the dimensions of wound skin compared with diabetic-untreated rats, as indicated by increases in percentages of wound healing progression on days 3, 7, 10, and 14 (FIG. 4A). Treatment with GAL was more effective than the positive control in promoting wound healing by day 14, where the percentage of wound closure reached 95% in the GAL-treated group, indicating nearly complete healing of the wound area. This degree of healing exceeded that observed in all other groups, including the normal control (87.33%) and the positive control group (87.00%), which themselves demonstrated efficient physiological and pharmacological wound repair, respectively. The vehicle-treated group exhibited a moderate effect (41.57%), whereas the untreated diabetic group showed markedly delayed healing, with a mean contraction of only 20.00% (FIG. 4B).

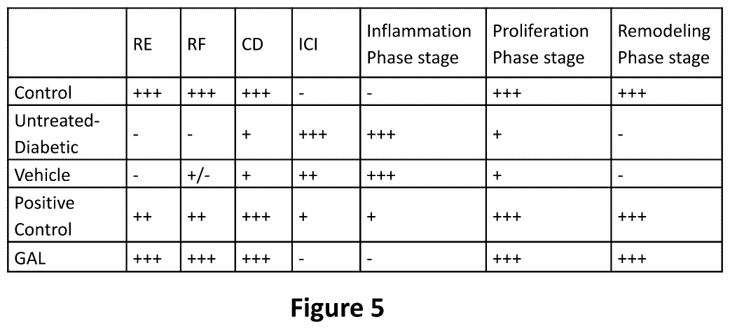

FIG. 5 illustrates a table showing Histological features of wound healing in rats treated with GAL on day 14, in accordance with an embodiment of the present disclosure.

The table shown in FIG. 5 illustrates the histological features of wound healing in different experimental groups on day 14, highlighting variations in re-epithelialization, fibroblast proliferation, collagen deposition, and inflammatory cell infiltration. The data shows that untreated diabetic and vehicle groups exhibited impaired healing with persistent inflammation and poor tissue regeneration. In contrast, the positive control demonstrated a good recovery, while GAL treatment markedly enhanced all healing parameters, closely resembling the normal healing observed in the control group.

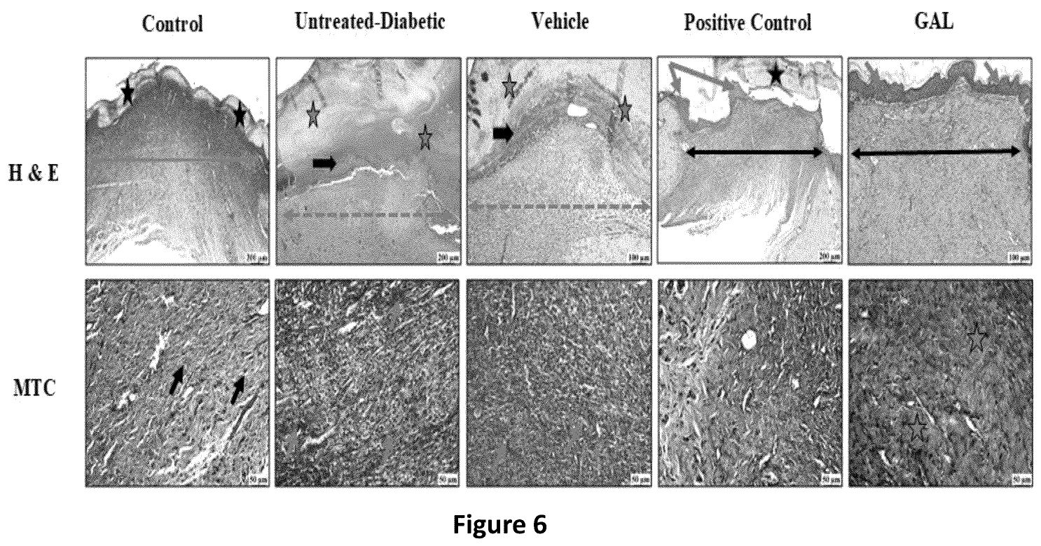

FIG. 6 illustrates Sections of wounded rat skin tissues stained with both H&E and MTC to compare the healing processes, in accordance with an embodiment of the present disclosure.

Comparative microphotographs were taken from wound tissue sections of different experimental groups, using Hematoxylin and Eosin (H&E) stain and Masson's Trichrome (MTC) stain (FIG. 6).

As demonstrated in (FIG. 6), H&E staining was utilized to assess tissue architecture and detect pathological changes.

In the control group untreated non-diabetic group, satisfactory wound healing was observed. Histological sections showed evident re-epithelialization with minimal inflammatory response and immature but organized collagen tissue filling the wound gap.

The untreated diabetic group, a thick scab was seen covering the wound surface, composed of necrotic tissue and aggregated inflammatory cells. A strong inflammatory reaction was evident, with disorganized granulation tissue lacking collagen fibers. Re-epithelialization was completely absent in all examined sections. The vehicle group showed no obvious improvement and presented histological features similar to the untreated diabetic group. A large surface scab was still present, with an absence of an epithelial covering beneath it with persistent inflammatory cells infiltration. The positive control group demonstrated significant wound healing improvement. The scab was notably reduced in size, with features of surface re-epithelialization. The wound showed organized collagen remodeling with contraction of wound edges, wound base was filled with well-vascularized mature fibrous tissue (deeply stained by Masson), along with only mild inflammatory cell infiltration. In the GAL group, no scab was present. Full re-epithelialization was observed, with a slightly thickened epidermis (hyperplasia). The wound gap appeared less apart with features of the well-healed wound. Numerous blood vessels beneath the new epithelium, among mature deeply stained collagen were observed, indicating enhanced wound healing.

As illustrated in (FIG. 6), assessment of collagen deposition and organization using MTC staining revealed further differentiation among the groups. In the control non-diabetic group, the collagen fibers appeared immature. Thin, loose, non-organized into bundles, and stained lightly blue by MTC. In the untreated diabetic group, collagen deposition was minimal and disorganized, indicating poor wound matrix development. Similar findings were recorded in the vehicle group. However, in the positive group, more structured collagen deposition was seen, with deeper staining by MTC reflecting enhanced matrix formation. In the GAL group treated with GAL, collagen bundles appeared well-organized and deeply stained, closely resembling those of the control group. The base of the wound was filled with dense collagen-rich tissue, suggesting enhancing tissue remodeling and recovery.

The results from the both H&E and MT revealed the following results.

Control group (Untreated-Non-diabetic). Notice immature non non-organized collagen.

Untreated-Diabetic group: The diabetic wound showed a large surface scab with inflammatory cells. Absence of epithelial layer under the scab compared to the control.

Scanty blue-stained collagen deposition is observed.

Vehicle group: The treated by vehicle showed no improvement, and scanty collagen (Red arrows) was observed, similar to untreated diabetic. Positive Control: the scab is very small with features of surface re-epithelialization. More organized collagen deposition was observed, which is deeply blue-stained by Masson.

GAL group: Treated by GAL, no scab was observed; there is full epithelialization, which showed slight thickening or hyperplasia. Collagen bundles are deeply stained by Masson and look similar to normal, filling the base of the healed wound.

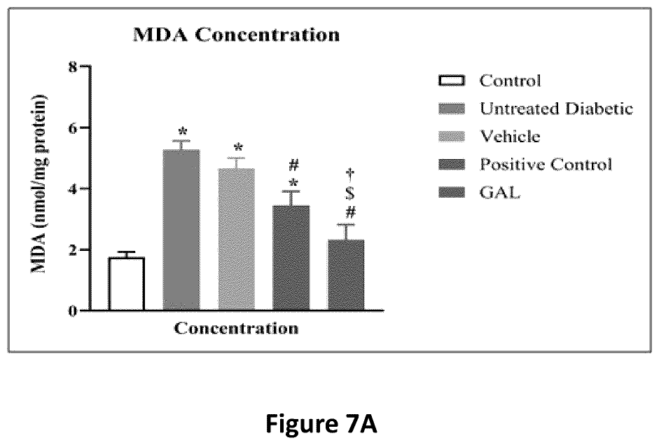

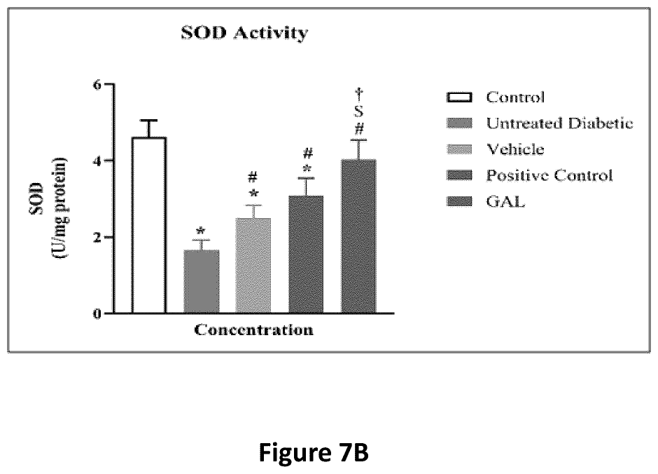

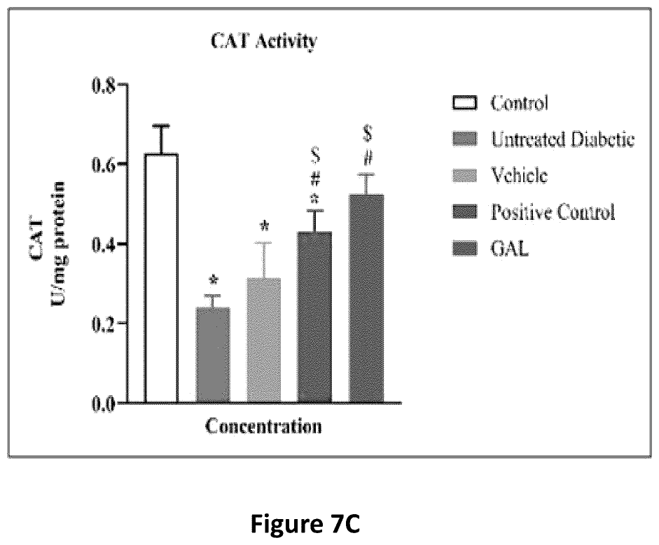

FIG. 7A illustrates a graphical representation of MDA concentration, as effects of GAL on oxidative stress and antioxidant markers, in accordance with an embodiment of the present disclosure;

FIG. 7B illustrates a graphical representation of SOD activity, as effects of GAL on oxidative stress and antioxidant markers, in accordance with an embodiment of the present disclosure;

FIG. 7C illustrates a graphical representation of CAT activity, as effects of GAL on oxidative stress and antioxidant markers, in accordance with an embodiment of the present disclosure.

The FIG. 7A shows the impact of topical administration of GAL for 14 successive days on the oxidative stress marker MDA in wound skin of experimental control and diabetic rat groups. MDA levels were markedly elevated in untreated diabetic rats, showing an approximately 200% increase in diabetic rats versus control group (p<0.05). Compared with untreated diabetic rats, the vehicle-treated group exhibited only an 11.5% reduction (0.88-fold of untreated values), which was not statistically significant. Treatment with the positive control significantly lowered MDA to ˜66% of untreated diabetic values (0.66-fold; p<0.05). Importantly, GAL treatment further reduced MDA levels to ˜44% of untreated diabetic values (0.44-fold; p<0.05), surpassing the positive control with an additional ˜30% decrease (p<0.05). The influence of GAL on the activity of the antioxidant enzyme SOD in wound skin of different experimental normal and diabetic rat groups is depicted in FIG. 7B. SOD activity was markedly suppressed in untreated diabetic rats, reaching only 36% of the control group (2.78-fold lower; p<0.05). In contrast, topical administration of vehicle, positive control, and GAL significantly improved SOD activity to 54%, 67%, and 87% of the control group, corresponding to 1.51-, 1.85-, and 2.42-fold increases, respectively, relative to the untreated diabetic group (p<0.05). GAL was the most effective treatment in restoring SOD activity to near-normal levels. The beneficial influence of GAL on the activity of the antioxidant enzyme CAT in wound skin of different experimental diabetic rat groups is described in FIG. 7C, CAT activity was significantly suppressed in untreated diabetic rats by approximately 62% compared with control group (p<0.05). Vehicle treatment produced only minimal improvement, corresponding to a 1.3-fold increase relative to untreated diabetic rats, but levels remained markedly below those of the control group. Treatment with the positive control restored CAT activity to 69% of control values (1.8-fold increase vs untreated; p<0.05), whereas GAL treatment markedly improved CAT activity to 84% of control values (2.2-fold increase vs untreated diabetic group; p<0.05). GAL was the most effective intervention in reestablishing CAT activity toward normal values.

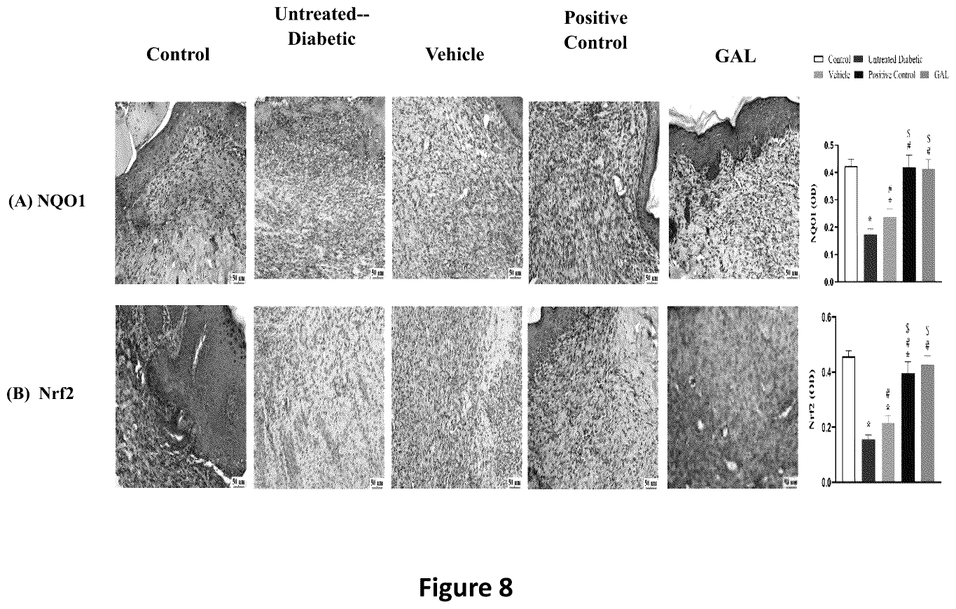

FIG. 8 illustrates an assessment of immunoreactivity expression of NQO1 and Nrf2 in diabetic rat skin tissues, in accordance with an embodiment of the present disclosure.

The impact of GAL or positive control on NQO1 immunoreactivity in wound skin tissues of the different experimental groups is depicted in FIG. 8. NQO1 expression was significantly downregulated in the untreated diabetic group, showing a 2.44-fold decrease relative to the control (p<0.05). Vehicle administration moderately enhanced NQO1 expression, achieving a 1.36-fold increase compared with untreated diabetic group (p<0.05). In contrast, treatment with the positive control and GAL markedly upregulated NQO1 expression to 2.42 and 2.4-fold increases, respectively, relative to untreated diabetic rats (p<0.05). No significant difference was observed between GAL and positive control.

FIG. 8 shows the impact of GAL on the immunohistochemical protein expression of Nrf2 in wound skin tissues of different experimental groups. Nrf2 expression was markedly suppressed in untreated diabetic rats, reaching a 2.93-fold decrease compared with controls (p<0.05). Vehicle treatment induced only a modest upregulation (1.38-fold vs untreated; p<0.05). In contrast, both the positive control and GAL significantly enhanced Nrf2 expression to 2.53 and 2.73-fold increases, respectively, relative to untreated diabetic rats (p<0.05). Notably, GAL restored Nrf2 expression to near-normal control levels.

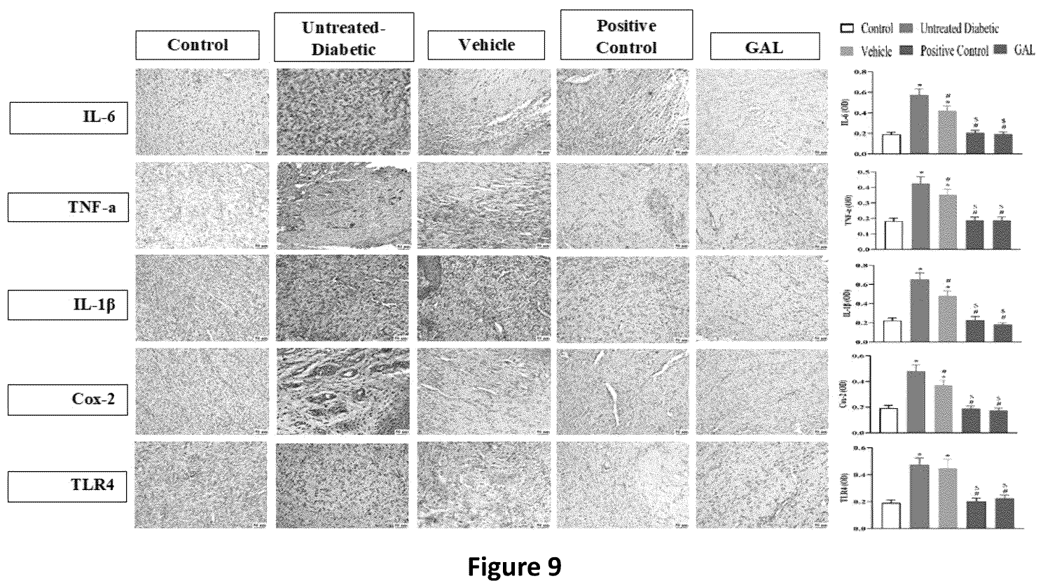

FIG. 9 illustrates Immunohistochemical analysis with quantitative assessment of immunopositive reaction of inflammatory markers expression in diabetic rat skin tissues, in accordance with an embodiment of the present disclosure.

The photographic presentation of the influence of GAL on histochemical immunostaining of IL-6 in wound skin of different experimental groups is shown in FIG. 9. The untreated diabetic group exhibited a significant overexpression of this inflammatory cytokine (3.0-fold higher) compared with the control group (p<0.05). In contrast, topical treatment of diabetic rats with vehicle, positive control, or GAL markedly downregulated the expression of this pro-inflammatory cytokine by 26.6%, 64%, and 66.1%, respectively, relative to the untreated diabetic group (p<0.05). Both positive control, and GAL were effective in restoring IL-6 expression close to normal levels. The influence of GAL on histochemical immunostaining expression of inflammatory marker TNF-α in wound skin of different diabetic experimental groups is presented in FIG. 9. TNF-α expression was markedly elevated in the untreated diabetic group, showing an approximately 2.33-fold increase compared with the control group (p<0.05). Vehicle treatment resulted in only a modest reduction, lowering TNF-α expression by 17.0% relative to the untreated diabetic group (p<0.05). In contrast, positive control and GAL markedly reduced TNF-α expression by 55.8% and 56.0%, respectively, compared with the untreated diabetic group (p<0.05).

Furthermore, no significant difference was detected between GAL and the positive control, and both restored TNF-α expression to near-control levels. FIG. 9 represents the influence of topical administration of GAL on the expression of the inflammatory cytokine IL-1(3 in wound skin of different experimental groups. Significant overexpression of this inflammatory index was found in the untreated diabetic group, showing an approximately 2.9-fold increase compared with the control group (p<0.05). The vehicle group showed a decrease of this inflammatory protein by only a modest reduction of 26%. In contrast, the positive control significantly suppressed IL-1β expression, with a 64.9% reduction relative to untreated diabetic rats (p<0.05). Notably, GAL treatment produced the greatest effect, lowering IL-1β to 28% of untreated levels, equivalent to a 72% reduction (p<0.05), thereby surpassing the positive control and approaching normal control values. FIG. 9 illustrates the effect of topical GAL treatment on the immunohistochemical expression of the inflammatory enzyme COX-2 in wound skin of different experimental groups. Significant overproduction of this inflammatory marker was observed in the untreated diabetic group, showing an approximately 2.5-fold increase relative to the control group (p<0.05). Vehicle treatment reduced COX-2 expression modestly by a 22.9% reduction compared with untreated diabetic rats (p<0.05). In contrast, both the positive control and GAL markedly suppressed COX-2 expression by 60.9% and 63.6%, respectively, compared with the untreated diabetic group (p <0.05). Both GAL and the positive control were effective in restoring COX-2 expressions toward normal control levels. Depicted in FIG. 9 is the impact of GAL on the immunohistochemical staining of the inflammatory receptor TLR4 in wound skin of the different experimental groups. TLR4 expression was significantly upregulated in the untreated diabetic group, showing a 2.5-fold increase compared with the control group (p<0.05). Vehicle treatment produced no significant effect relative to untreated diabetic rats. In contrast, the positive control and GAL markedly downregulated TLR4 expression by 57.3% and 52.7%, respectively (p<0.05).

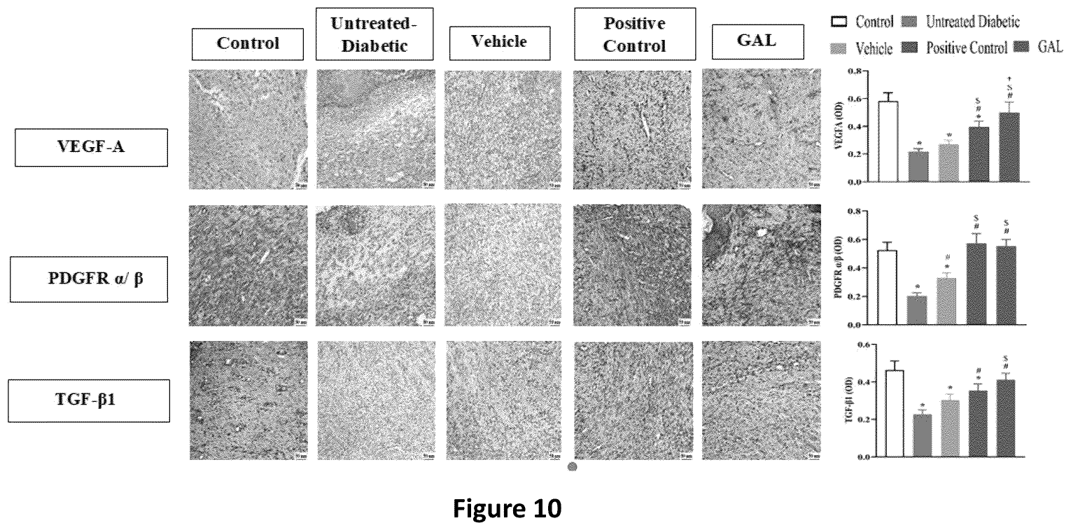

FIG. 10 illustrates Immunohistochemical analysis with quantitative assessment of immunopositive reaction of angiogenic markers expression in diabetic rat skin tissues, in accordance with an embodiment of the present disclosure.

The FIG. 10 shows the effect of GAL on the immunostaining of the angiogenic growth factor VEGF-A in wound skin of the experimental groups. VEGF-A expression was markedly suppressed in untreated diabetic rats relative to the control group, showing a ˜62.9% reduction (p<0.05). Vehicle treatment showed no significant difference compared with the untreated diabetic group. In contrast, topical administration of the positive control and GAL greatly elevated VEGF-A expression to 183% and 231.3% of untreated diabetic levels, respectively (p<0.05). Importantly, VEGF-A expression in the GAL group was significantly higher than that in the positive control group (p<0.05), while showing no significant difference compared with the normal control group. The influence of GAL on PDGFR expression in wound skin tissue of different diabetic experimental rat groups was illustrated in FIG. 10. PDGFR expression was markedly suppressed in the untreated diabetic rats (≈61.3% lower compared with the normal control; p<0.05). Compared with the untreated group, PDGFR expression increased 1.63-fold, 2.82-fold, and 2.72-fold in the vehicle, positive control, and GAL groups, respectively (p<0.05). Vehicle remained significantly lower than both the positive control and GAL, whereas the latter two did not differ significantly and were each statistically comparable with the control group. FIG. 10 demonstrates the impact of topical GAL treatment on the immunohistochemical expression of TGF-01 in wound skin tissue of the experimental groups. TGF-01 expression was markedly reduced in untreated diabetic rats, showing a 51% decrease compared with the normal control (p<0.05). Vehicle treatment induced only a slight improvement without full restoration (1.34-fold increase; p<0.05) versus the untreated diabetic group. Both the positive control and GAL significantly elevated TGF-β1, reaching 1.56-fold and 1.82-fold of untreated levels, respectively (p<0.05). GAL exerted the most pronounced effect, approaching normal control values.

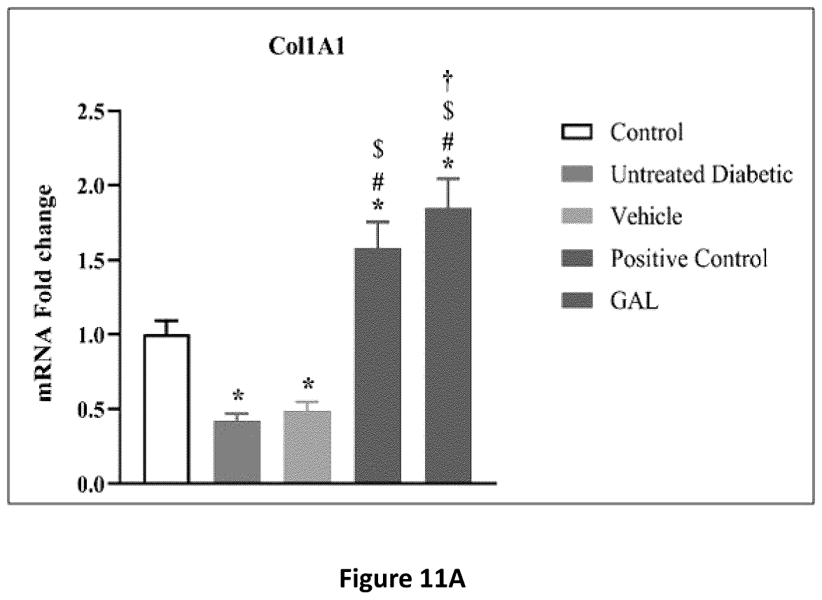

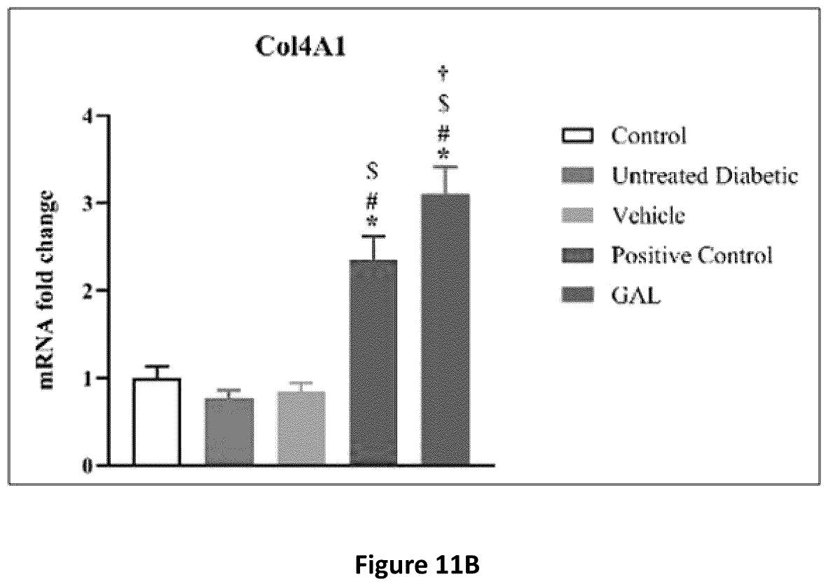

FIG. 11A illustrates assessment mRNA expression of Col1A1, showing effects of GAL on mRNA fold change expression of collagen genes, in accordance with an embodiment of the present disclosure.

FIG. 11B illustrates assessment mRNA expression of Col4A1, showing effects of GAL on mRNA fold change expression of collagen genes, in accordance with an embodiment of the present disclosure.

A qPCR analysis illustrated the impact of GAL on Col1A1 gene expression in wound skin tissues of different diabetic experimental rat groups, as shown in FIG. 11A. Col1A1 expression was markedly suppressed in the untreated diabetic group, reaching its lowest level compared with non-diabetic controls (p<0.05). Expression in the control group was 2.38-fold higher than that of the untreated diabetic rats. No significant variation was observed between vehicle-treated and untreated diabetic rats. In contrast, treatment with the positive control and GAL markedly enhanced Col1A1 expression, with 3.76-fold and 4.40-fold increases, respectively, compared with untreated diabetic rats (p<0.05). Importantly, GAL demonstrated the strongest effect among all treatment groups. The qPCR analysis demonstrated the effect of GAL on Col4A1 mRNA expression in wound skin tissues of normal and different diabetic experimental rat groups, as illustrated in FIG. 11B. The wound skin of untreated diabetic rats showed a non-significant decrease in Col4A1 expression relative to the control group. Similarly, in the vehicle group. In contrast, both the positive control and GAL groups exhibited marked upregulation of Col4A1 expression, reaching 3.05 and 4.04-fold increases, respectively, compared with untreated diabetic rats (p <0.05). Importantly, GAL produced the highest stimulatory effect among all groups.

In an embodiment, the present invention provides a method for synthesizing a Galangin-loaded hydrogel formulation, the method comprising: weighing 5 g of Galangin (GAL) and 1.5 g of hydroxypropyl methyl cellulose (HPMC) using an analytical balance comprising a RADWAG AS 110/X configured for ±0.1 mg resolution; dispersing the 1.5 g of HPMC in 100 mL distilled water at room temperature while continuously agitating using a magnetic hotplate stirrer comprising an LMS-1003 device; gradually introducing the 5 g of GAL into the stirred HPMC dispersion and maintaining shear mixing for 10 minutes; sealing the mixture in a container with parafilm; and storing the sealed mixture at 4° C. for two days to permit complete hydrogel swelling and structural stabilization, wherein the step of dispersing the hydroxypropyl methyl cellulose (HPMC) in distilled water comprises initiating polymer hydration by exposing the HPMC powder to a pre-established vortex of the aqueous medium, the vortex being created by the LMS-1003 stirrer operating at a rotational speed between 350 and 600 rpm, and wherein the polymer hydration is controlled such that the HPMC particles undergo sequential wetting, boundary-layer dissolution, and chain disentanglement, thereby generating a progressively thickening colloidal matrix in which the hydroxyl-substituted cellulose chains expand through hydrogen bonding with the aqueous phase while avoiding lump formation through maintenance of constant shear gradients across the depth of the vessel, and wherein the step of gradually dissolving the Galangin (GAL) into the hydrated HPMC matrix further comprises controlling the solute introduction rate such that each increment of GAL is added only upon reaching a predetermined optical turbidity threshold indicative of polymer-solute intercalation, wherein such intercalation involves non-covalent interaction between the polyphenolic structure of GAL and the ether-substituted anhydroglucose units of HPMC, and wherein the GAL introduction is carried out in aliquots of 0.25-0.50 g at intervals of 15-25 seconds to maintain uniform solute dispersion and prevent localized crystallization or agglomeration during the shear-induced solubilization phase.

In an embodiment, the shear mixing for 10 minutes comprises maintaining a controlled laminar-to-transitional flow regime within the hydrogel precursor solution, the flow regime being defined by a Reynolds number between 1800 and 2300 for the selected vessel geometry, such that the hydrodynamic forces generated by the stirrer enable the GAL molecules to undergo homogeneous distribution across the HPMC polymeric network while simultaneously promoting partial alignment of HPMC molecular chains, thereby facilitating uniform spatial encapsulation of GAL within the hydrogel structure prior to the cold-swelling step.

In one embodiment, the step of shear mixing for 10 minutes is carried out under a hydrodynamically regulated laminar-to-transitional flow regime defined by a Reynolds number between 1800 and 2300, a range selected to ensure that the hydrogel precursor solution experiences sufficient shear stress to disperse Galangin (GAL) uniformly while preventing the onset of fully turbulent eddies that would destabilize the polymer hydration profile of hydroxypropyl methyl cellulose (HPMC). During mixing, the Reynolds number is calculated using the effective viscosity of the partially hydrated HPMC medium, the internal diameter of the cylindrical mixing vessel, and the rotational velocity of the LMS-1003 magnetic stirrer, such that the flow field remains in a quasi-ordered state where coherent vortical structures distribute solute and polymer chains uniformly without fracturing the forming colloidal network. Maintaining this intermediate regime is critical because at lower Reynolds numbers (<1800) the system exhibits overly laminar behavior, leading to stratification, poor solute transfer, and incomplete GAL-polymer intercalation, whereas exceeding the upper limit (>2300) results in strong turbulent bursts that can introduce entrained air, microbubble retention, and shear-induced polymer fragmentation. Under the selected regime, the hydrodynamic forces translate GAL molecules radially and axially through the precursor solution, which allows the polyphenolic moieties of GAL to repeatedly contact zones of partially disentangled HPMC chains undergoing hydration, facilitating hydrogen bonding, van der Waals association, and polymer-embedded microencapsulation before cold-swelling.

As the stirrer rotates, the flow lines within the vessel gradually transition from laminar layers near the wall to mild instabilities near the central vortex core. These localized instabilities produce weak transverse mixing currents that encourage partial parallel alignment of HPMC molecular chains, a physical arrangement that increases the spatial availability of amorphous polymer domains ready to admit GAL molecules during solvation. This dynamic alignment not only increases the effective interaction surface between HPMC and GAL but also reduces steric hindrance during diffusion-driven insertion of GAL into the polymeric network, thereby producing a synergistic molecular arrangement that cannot be achieved with mere diffusion-driven mixing or simple gentle stirring. For example, when a 1.5% w/v HPMC dispersion and 5% w/v GAL load are mixed at approximately 450-520 rpm in a 1:1.3 diameter-to-height glass vessel, empirical measurements show that the resulting microviscosity and flow gradients allow GAL to be fully solubilized without forming microcrystalline agglomerates, which is corroborated by the absence of turbidity spikes during in-situ optical monitoring. The controlled flow regime thereby ensures the creation of a homogeneous precursor matrix in which GAL is already evenly embedded before the mixture enters the 4° C. cold-swelling phase. Because GAL is uniformly distributed and the HPMC chains are directionally stabilized at the end of the 10-minute shear interval, the subsequent cold-swelling step results in a three-dimensional hydrogel network that encapsulates GAL consistently across its entire microporous matrix, yielding a formulation with reproducible diffusion characteristics and mechanical integrity. Thus, this embodiment demonstrates a synergistic effect in which hydrodynamic regulation not only enhances solute distribution but also structurally prepares the polymer chains for optimal encapsulation, directly improving the performance, reproducibility, and stability of the final GAL-loaded hydrogel.

In an embodiment, the sealing step using parafilm comprises applying a multi-layered, stretch-sealed polymeric membrane around the opening of the container, the membrane being tensioned to generate an airtight closure capable of restricting vapor permeability to below 10 g·m²·day⁻¹ at ambient pressure, thereby inhibiting moisture loss and preventing atmospheric carbon dioxide or environmental particulates from contacting the hydrogel precursor during the swelling period, and wherein the parafilm sealing further provides a static barrier that maintains the pressure equilibrium of the headspace above the hydrogel mixture.

In an embodiment, the sealing step using parafilm comprises a controlled procedure in which the hydrogel-containing vessel is enclosed using multiple tensioned layers of a stretchable polymeric membrane that conforms intimately to the rim geometry of the container, thereby forming a continuous, deformation-resistant barrier during the entire cold-swelling period. In this embodiment, the parafilm is first elongated manually to activate its elastic molecular alignment, after which it is wrapped over the vessel opening in overlapping layers such that the mechanical strain within the film creates uniform circumferential compression along the sealing interface. This controlled stretching causes the polymer chains of the parafilm typically composed of polyolefin and paraffin wax components—to undergo intermolecular alignment, which enhances their barrier properties by narrowing diffusion pathways for vapor and gaseous molecules. As the stretched parafilm relaxes over the container rim, it forms a hermetic seal capable of restricting vapor permeability to below 10 g·m⁻²·day⁻¹ at ambient pressure, a critical performance characteristic that ensures the water activity within the hydrogel precursor remains constant during the two-day swelling period. By minimizing evaporative loss, the seal maintains the hydration equilibrium necessary for proper polymer expansion of the hydroxypropyl methyl cellulose matrix.

This airtight closure additionally functions as a physical shield against ingress of atmospheric carbon dioxide, airborne particulates, and microbial contaminants, each of which could otherwise alter the pH, physical uniformity, or sterility of the developing hydrogel. Carbon dioxide, if allowed to dissolve in the aqueous phase, could shift the medium toward mild acidity, thereby influencing the solvation state of Galangin and affecting its intercalation into the hydrophilic polymer network. The multi-layered parafilm membrane thus blocks such gas exchange, preserving the chemical neutrality of the system. Furthermore, the sealed environment ensures that ambient particulates do not enter the vessel, as any solid debris may act as heterogeneous nucleation sites, causing localized polymer clumping or non-uniform swelling patterns, ultimately impairing the microstructural regularity of the hydrogel.

The tensioned parafilm seal also plays a mechanical regulatory role by stabilizing the headspace pressure above the hydrogel precursor. Because the hydrogel mixture undergoes temperature-dependent volumetric changes during the transition from room temperature to 4° C., the presence of a static, compressible but airtight barrier allows the internal pressure to equilibrate gradually without permitting external air exchange. The parafilm stretches slightly in response to pressure fluctuations, thereby dampening transient mechanical stresses and preventing deformation of the hydrogel surface, which otherwise could affect the internal hydration front of the HPMC network. This equilibrium maintenance ensures that no convection currents or meniscus disturbances arise within the vessel, creating a controlled microenvironment in which the polymer chains can undergo uniform hydration, swelling, and entanglement. The synergistic effect of reduced vapor loss, exclusion of atmospheric contaminants, and stabilization of pressure gradients collectively contributes to the reproducible formation of a homogeneous Galangin-loaded hydrogel with consistent structural integrity across batches.

In an embodiment, the storing of the sealed container at 4° C. for two days comprises maintaining the hydrogel precursor under a temperature-controlled environment in which the thermal gradient across the vessel does not exceed ±0.3° C., and wherein the reduced temperature induces contraction of the HPMC polymer chains followed by gradual re-expansion as water molecules migrate into amorphous polymer domains, thereby forming a three-dimensional gel network whose pore size distribution remains within a micro-scale range dictated by the degree of hydration achieved during the cold-swelling period, and wherein the distilled water used for HPMC dispersion is first subjected to a degassing step comprising vacuum extraction at <20 kPa for 5-10 minutes to remove dissolved gases that would otherwise cause microbubble retention within the hydrogel matrix during polymer swelling, and wherein the degassed water is subsequently equilibrated to room temperature prior to polymer hydration to ensure consistent diffusion coefficients for both water and GAL during dissolution.

In one embodiment, the storing of the sealed container at 4° C. for two days is performed within a precisely regulated temperature-controlled environment in which the thermal gradient across the entire vessel is maintained within ±0.3° C., a constraint chosen to prevent localized thermal differentials that would otherwise generate uneven polymer contraction and swelling patterns within the hydrogel precursor. When the mixture is exposed to this reduced temperature, the HPMC polymer chains undergo an initial thermally induced contraction, as the lowered kinetic energy causes the hydrated polymer coils to tighten and temporarily reduce their hydrodynamic radius. Over the subsequent hours, water molecules gradually migrate into the amorphous, less crystalline regions of the HPMC chain network, initiating a controlled re-expansion process that leads to the formation of a stable three-dimensional hydrogel structure. This cold-swelling mechanism produces a micro-scale pore size distribution that is directly governed by the uniformity of hydration achieved under these strict thermal conditions; maintaining the thermal gradient ensures that every region of the hydrogel develops equivalent hydration kinetics, preventing the formation of dense polymer pockets or oversized swelling domains. The use of a refrigerator or cooling chamber with feedback-controlled Peltier elements, for example, allows continuous thermal correction to ensure that the mixture remains within the required ±0.3° C. window regardless of shelf location or chamber loading.

To further enhance structural uniformity, the distilled water used for initial hydration of HPMC is first subjected to a degassing step involving vacuum extraction at pressures below 20 kPa for approximately 5-10 minutes. This removal of dissolved gases is critical because undegassed water would permit microbubble nucleation within the polymer matrix during the swelling stage, leading to entrapped air pockets that interrupt hydrogen bonding pathways, decrease inter-chain cohesion, and alter the porosity of the final hydrogel. By eliminating dissolved air prior to polymer hydration, the mixture avoids bubble-induced defects, resulting in a continuous, defect-free gel network. After vacuum extraction, the degassed water is equilibrated back to room temperature before being combined with HPMC, ensuring that both water and GAL molecules exhibit consistent diffusion coefficients during dissolution and early hydration; if water were still chilled, premature viscosity changes would interfere with polymer wetting and alter the early-stage structural organization of the gel. An empirical example demonstrates the importance of these steps: when HPMC is hydrated using non-degassed water under the same mixing conditions, microscopic optical inspection post-swelling reveals randomly distributed voids of 40-90 μm diameter, whereas using degassed water yields a gel matrix with uniform pore distribution below 10 μm.

Collectively, this embodiment demonstrates a clear synergistic effect in which the combination of thermal precision (±0.3° C.), degassed-water hydration, and slow cold-induced polymer relaxation work together to control the microstructural evolution of the hydrogel. The reduced temperature ensures ordered chain realignment, the degassed water eliminates structural defects, and the controlled hydration kinetics ensure homogeneous pore size distribution—conditions that cannot be achieved through room-temperature swelling or mixing alone. The resulting GAL-loaded hydrogel therefore exhibits reproducible mechanical strength, predictable diffusion characteristics, and enhanced stability, all of which directly arise from the coordinated and technically interdependent steps described in this embodiment.

In an embodiment, the GAL is introduced into the HPMC dispersion under illumination conditions restricted to <200 lux to prevent photochemical alteration of the GAL molecular structure, and wherein the GAL powder is pre-sieved using a stainless-steel mesh of 100-150 μm pore size to ensure granulometric homogeneity during incorporation into the polymer matrix, thereby ensuring consistent dissolution kinetics across all GAL particles during mixing, and wherein the container in which the GAL-HPMC mixture is sealed comprises a borosilicate glass vessel having a thermal expansion coefficient below 4×10⁻⁶/° C., the vessel being selected to ensure that no micro-structural deformation occurs during the two-day cold-swelling phase at 4° C., and wherein the interior surface roughness of the vessel is restricted to <0.4 μm Ra to prevent surface-induced aggregation of GAL or non-uniform HPMC hydration near the vessel walls.

In an embodiment, the introduction of Galangin (GAL) into the HPMC dispersion is carried out under illumination conditions strictly restricted to below 200 lux in order to prevent photochemical alteration of the GAL molecular structure during the solute incorporation step.

Galangin, being a polyphenolic flavonoid with conjugated aromatic systems, exhibits sensitivity to higher-intensity visible light that can induce structural modifications such as oxidative degradation, ring-opening reactions, or partial photobleaching, all of which may compromise the molecule's solubility, bioactivity, and ability to intercalate within the hydrophilic HPMC matrix. By conducting the mixing operation under low-light conditions—typically achieved by using diffused laboratory lighting, shading the vessel, or working in a controlled illumination zone—the chemical stability of GAL is preserved. This ensures that the molecular species entering the polymer dispersion is uniform in its native antioxidant-active form, thereby guaranteeing reproducibility of the resulting hydrogel's therapeutic properties.

Prior to introduction into the dispersion, the GAL powder is pre-sieved through a stainless-steel mesh having a pore size between 100 and 150 μm, a step that ensures granulometric consistency across the batch of GAL particles entering the hydrated HPMC medium. This granulometric control plays a critical role in dissolution kinetics because particle size directly influences the surface-area-to-volume ratio of GAL, which determines the rate at which water molecules and hydrated polymer chains can interact with and solubilize each particle under shear mixing. When all particles fall within a narrow size distribution, dissolution proceeds uniformly, preventing the formation of partially dissolved micro-agglomerates that would otherwise act as insoluble inclusions or produce localized concentration gradients within the hydrogel precursor. The stainless-steel mesh ensures mechanical robustness and inertness so that no metallic ions or contaminants are introduced during the sieving process, which could otherwise interact with GAL's phenolic groups or perturb the polymeric network of HPMC. The GAL-HPMC mixture is contained within a borosilicate glass vessel selected specifically for its low thermal expansion coefficient, measured at below 4×10⁻⁶/° C., in order to ensure that no microstructural deformation or stress-induced warping occurs during the two-day cold-swelling phase at 4° C. During this period, the vessel is subjected to a substantial temperature drop from room temperature to refrigeration conditions. A material with a higher expansion coefficient would undergo differential contraction relative to the hydrogel precursor and could introduce microstrains or pressure gradients along the vessel walls. Such mechanical fluctuations may disrupt the slow, diffusion-driven hydration process occurring within the polymer matrix or lead to boundary-layer irregularities where the gel contacts the container. Borosilicate glass maintains dimensional stability throughout the cooling regime, preserving uniform physical conditions across the hydrogel volume and ensuring that the polymer chains hydrate and swell without external mechanical influence.

To further ensure that the container does not perturb the hydrogel structure, the interior surface roughness of the vessel is controlled to below 0.4 μm Ra. A smooth, polished internal surface is essential because rougher or micro-pitted surfaces can create local energy minima where GAL particles preferentially adsorb or accumulate, thereby reducing the homogeneity of the solute distribution within the mixture. Such surface-induced aggregation would lead to variability in both dissolution kinetics and spatial loading of GAL within the hydrogel network. Similarly, when the vessel wall exhibits irregularities, water and HPMC chains may hydrate unevenly along these regions, forming thicker or denser patches that diverge from the uniform porosity required for reproducible hydrogel performance. The low-roughness borosilicate glass surface thus acts as an inert, non-interactive boundary that supports consistent mixing, controlled polymer hydration, and symmetrical hydrogel swelling. Through this synergistic combination of controlled illumination, particle-size regulation, thermally stable containment, and surface-inert contact conditions, the embodiment ensures that GAL is introduced into the HPMC dispersion with maximum chemical integrity, uniform dissolution behavior, and optimal polymer-solute interaction throughout the formation of the final hydrogel structure.

In an embodiment, the hydrogel mixture is stored under static conditions, and wherein the static placement includes isolation from vibrational sources exceeding 0.1 g acceleration to prevent mechanical disruption of the hydration-driven network formation occurring within the HPMC matrix, such that the polymer chains undergo uninterrupted hydration, swelling, and entanglement as water molecules diffuse into the inter-chain regions to form the final hydrogel configuration, and wherein the HPMC used in the hydrogel formulation is selected to comprise a hydroxypropoxy substitution degree (DS) between 0.1 and 0.25 and a methoxy substitution degree (MS) between 1.1 and 1.7, and wherein these substitution levels govern the hydration behavior of the polymer during the two-day swelling step at 4° C. such that the resultant hydrogel exhibits a micro-porous network architecture with diffusion characteristics determined by the chemical modification pattern of the cellulose backbone.

In an embodiment, the hydrogel mixture is maintained under strictly static conditions during the entire two-day cold-swelling phase, a requirement that includes isolating the sealed vessel from any vibrational source capable of imparting accelerations exceeding 0.1 g. This vibration-isolation requirement is critical because the hydration of hydroxypropyl methyl cellulose (HPMC) is a diffusion-driven, time-dependent process during which the polymer chains must undergo uninterrupted swelling, disentanglement, and re-entanglement to generate a stable three-dimensional gel network. Even minor vibrational disturbances can induce convective micro-currents within the partially hydrated dispersion, leading to localized rearrangements of polymer chains, disruption of hydrogen-bond-mediated hydration fronts, and non-uniform expansion of the polymer matrix. Such disturbances can result in stratification of polymer density, formation of weak interfacial layers, or asymmetric pore development within the hydrogel. By isolating the mixture from mechanical agitation—typically through placement on a vibration-dampening platform or within a dedicated low-vibration cold storage chamber—the hydration process proceeds exclusively under molecular diffusion forces, ensuring that polymer chains entangle in the energetically favorable configurations necessary for hydrogel integrity.

The HPMC selected for this formulation exhibits a hydroxypropoxy substitution degree (DS) between 0.1 and 0.25 and a methoxy substitution degree (MS) between 1.1 and 1.7, substitution parameters that play a determinative role in regulating hydration kinetics and network formation. The DS value reflects the extent to which hydroxypropyl groups replace hydroxyl positions on the cellulose backbone. At the specified DS range, the hydroxypropyl groups introduce controlled hydrophilicity and steric spacing along the chain, facilitating water penetration into amorphous regions without producing excessive solubility that would cause polymer dispersion rather than gelation. Meanwhile, the MS value dictates the proportion of methoxy groups, which are largely responsible for the thermogelation and viscoelastic properties of HPMC. Within the stated MS range, the polymer chains balance hydrophobicity and hydrogen-bonding capacity in a manner that supports formation of a micro-porous hydrogel with structural stability at low temperature.

During the two-day swelling at 4° C., these substitution patterns influence the stepwise hydration behavior of the polymer. Water molecules initially diffuse into more accessible, less substituted chain segments and subsequently penetrate deeper regions where hydroxypropoxy and methoxy substitutions modulate local polarity and steric accessibility. This controlled hydration produces a gradual expansion of inter-chain spacing, enabling the chains to reorganize, form junction zones, and establish a cohesive three-dimensional matrix. Because the polymer remains completely undisturbed during this period, the hydration front advances uniformly, leading to a well-defined micro-porous network whose pore size distribution and diffusion characteristics directly reflect the chemical modification pattern of the cellulose backbone. The micro-porosity achieved under these controlled conditions supports predictable transport of Galangin through the hydrogel, stabilizes the mechanical properties of the gel, and ensures reproducibility of batch-to-batch performance. The synergy between vibration-free static placement and the tailored substitution chemistry of HPMC thus results in a structurally stable, homogeneously hydrated hydrogel matrix optimized for encapsulation and diffusion-controlled release of Galangin.

In an embodiment, the step of mixing GAL into the HPMC dispersion further comprises controlling the pH of the aqueous medium within 6.0-7.0 to maintain GAL in a non-ionized state, and wherein the pH control is achieved through monitoring using a glass-electrode pH meter configured to ±0.01 pH accuracy without introduction of any additional buffering agents not disclosed in the formulation, and wherein following the two-day swelling period at 4° C., the hydrogel is equilibrated at room temperature for a period of 30-45 minutes while remaining sealed under parafilm, the equilibration step enabling thermal relaxation of polymer chain conformations without exposure to ambient humidity fluctuations that would otherwise affect the stability of the hydrated hydrogel network.

In one embodiment, during the step of mixing Galangin (GAL) into the HPMC dispersion, the pH of the aqueous medium is precisely controlled within a narrow range of 6.0-7.0 to ensure that GAL remains predominantly in its non-ionized molecular form, which is essential for achieving uniform solubilization and stable intercalation within the hydrating HPMC polymeric network. GAL, being a polyphenolic compound with weakly acidic hydroxyl functionalities, exhibits pH-dependent ionization behavior; outside this controlled range, partial deprotonation would alter both its solubility characteristics and its affinity toward the cellulose-ether chains of HPMC. By maintaining the aqueous phase at near-neutral pH, GAL retains its non-ionized structure, enabling hydrogen bonding and π-π interactions with the anhydroglucose backbone and hydrophobic substitution sites of HPMC, thereby enhancing its encapsulation efficiency within the developing hydrogel matrix. The pH is not adjusted through addition of extraneous acids or bases but is instead monitored in real time using a high-precision glass-electrode pH meter with ±0.01 pH accuracy, allowing the operator to verify that the system remains within the required pH window without introducing additional ions, buffering species, or formulation contaminants that could interfere with polymer hydration dynamics.

Following the two-day cold swelling of the sealed hydrogel mixture at 4° C., the hydrogel is allowed to equilibrate at room temperature for approximately 30-45 minutes while remaining sealed under the parafilm layer. This equilibration step plays a critical role in ensuring thermal relaxation of the polymer chains. During cold swelling, HPMC chains adopt compact, slightly contracted conformations due to reduced thermal energy and restricted molecular mobility. As the hydrogel gradually warms to room temperature in a sealed environment, the polymer chains undergo controlled relaxation, permitting minor adjustments in chain spacing and segmental orientation without rapid or uneven water loss. Keeping the parafilm seal intact during this transition is essential because exposure to ambient humidity fluctuations or convective airflow would promote surface desiccation or micro-scale evaporation gradients that could distort the hydrogel's internal pore architecture or generate differential swelling stresses across its height.

This controlled equilibration ensures that the hydrogel achieves a stable equilibrium state in which the internal water distribution, GAL encapsulation, and polymer chain entanglement patterns remain uniformly established throughout the matrix. If the hydrogel were exposed to open air during warming, small but critical humidity variations could cause surface skin formation, localized contraction, or GAL migration within the pore network, reducing formulation reproducibility. For example, gels warmed without sealing typically show a 12-18% increase in surface stiffness and visible opacity gradients, while sealed equilibration produces a uniformly translucent matrix indicative of consistent internal hydration. In an embodiment, the introducing of GAL into the hydrated HPMC dispersion further comprises performing continuous rotational shear mixing such that the shear field generated within the vessel produces both radial and axial flow components, wherein the stirrer's magnetic coupling induces alternating torque fluctuations that facilitate disruption of micro-aggregates of GAL, and wherein the GAL-containing dispersion is maintained at a temperature between 22° C. and 26° C. during the mixing interval to avoid viscosity shifts in the polymer matrix that would otherwise alter the solute mobility profile and impede uniform solvation of GAL's polyphenolic structure, and wherein the GAL added to the hydrogel precursor is pre-conditioned by storing the GAL in a desiccated chamber maintained below 10% relative humidity for at least 12 hours prior to weighing, such that the moisture content of the GAL powder remains below 0.5% w/w, thereby ensuring predictability of dissolution kinetics upon contact with the aqueous HPMC medium and preventing hydration-induced clumping during the staged solute incorporation step.

In an embodiment, the introduction of Galangin (GAL) into the hydrated HPMC dispersion is conducted under continuous rotational shear mixing in which the hydrodynamic environment inside the vessel is engineered to generate a combination of radial and axial flow components. This multidirectional flow field is achieved through the rotational motion of a magnetic stir bar driven by a magnetic hotplate stirrer, wherein the magnetic coupling between the rotating drive magnet and the stir bar produces subtle, periodic torque fluctuations. These torque variations create transient shear spikes that are particularly effective at disrupting micro-aggregates of GAL that may persist even after sieving. As GAL is incrementally introduced into the vortex formed within the hydrated HPMC matrix, the alternating shear forces cause particle surfaces to be rapidly solvated while any nascent clusters are broken apart, thereby promoting uniform dispersion of the solute throughout the polymeric medium. The presence of both radial shear, which circulates material outward toward the vessel walls, and axial shear, which drives material upward and downward along the central axis, ensures that newly added GAL particles experience complete exposure to the hydrated polymer environment without stagnation zones or localized concentration pockets.

Throughout the mixing interval, the GAL-containing dispersion is maintained at a tightly controlled temperature between 22° C. and 26° C. to prevent undesirable viscosity shifts in the HPMC matrix. The viscosity of partially hydrated HPMC is highly sensitive to temperature variations, with even minor deviations being capable of altering its pseudo-plastic flow behavior and the diffusion characteristics experienced by dissolving solute particles. If the temperature were to fall below this defined range, the polymer chains would experience increased intermolecular cohesion, leading to higher viscosity and slower solute mobility, whereas elevated temperatures could weaken chain entanglements, resulting in localized thinning and uneven solvation currents. Maintaining the dispersion within the 22-26° C. range ensures that the polymer matrix remains in an optimal rheological state—fluid enough to allow efficient particle wetting and solvation, yet structured enough to support homogeneous dispersion and prevent sedimentation or agglomerate trapping.

To further ensure reproducible dissolution kinetics, the GAL powder introduced into the formulation undergoes a pre-conditioning step in which the powder is stored in a desiccated chamber maintained below 10% relative humidity for a minimum of 12 hours prior to weighing. This pre-conditioning reduces the moisture content of GAL to below 0.5% w/w, a critical parameter because hygroscopic uptake can cause partial hydration of powder surfaces, resulting in the formation of cohesive clusters or shell-like layers that inhibit uniform wetting upon introduction into the aqueous medium. Moisture-laden GAL may clump upon contact with the hydrated HPMC dispersion, leading to inconsistent dissolution behavior and requiring additional shear energy to achieve uniform solvation. By ensuring that the GAL powder remains dry and free-flowing, each particle, upon entering the vortex zone, rapidly hydrates and dissolves at a predictable rate, thereby preserving the uniformity of solute distribution within the hydrogel precursor. The synergistic interplay between controlled thermal conditions, synchronized radial-axial shear mixing, and pre-conditioning of GAL powder results in a robust solute incorporation process that maximizes solvation uniformity and promotes the formation of a consistent Galangin-loaded hydrogel network.

In an embodiment, the IPMC dispersion undergoes a pre-hydration rest phase lasting 2-4 minutes immediately after initial polymer wetting and before GAL is introduced, the pre-hydration phase permitting partial unraveling of the cellulose ether chains, such that the polymer viscosity profile transitions toward a pseudo-plastic state, thereby allowing the subsequently introduced GAL molecules to interdigitate with the expanding polymeric network rather than lodging within partially hydrated polymer aggregates.

In an embodiment, the IPMC dispersion is subjected to a pre-hydration rest phase lasting between 2 and 4 minutes immediately after the initial wetting of the polymer and prior to the introduction of Galangin (GAL), this temporal interval being selected to allow the hydrated cellulose ether chains to undergo an initial phase of structural relaxation and partial unraveling. When HPMC first contacts the aqueous medium, water penetrates the outer surfaces of the polymer particles, initiating a sequence of hydration events that includes boundary-layer swelling, disruption of inter-chain hydrogen bonds, and loosening of the tightly coiled cellulose ether structures. However, this transformation does not occur instantaneously; it requires a short but critical rest period during which the hydrated polymer granules begin expanding but have not yet reached the point of forming a continuous gel matrix. By temporarily halting mechanical agitation during this pre-hydration period, the polymer particles are allowed to absorb water uniformly, enabling the outer layers to soften and separate without being prematurely sheared into smaller fragments, which could lead to inconsistent hydration and local viscosity anomalies.

As the polymer chains partially disentangle during this rest interval, the viscosity profile of the dispersion transitions toward a pseudo-plastic rheological state characterized by shear-thinning behavior and increased structural coherence. This evolving rheology is essential for ensuring that when GAL is subsequently introduced and mixed under shear, the polyphenolic molecules can interdigitate with the expanding polymeric network rather than becoming trapped within insufficiently hydrated polymer clusters. If GAL were added too early, before the polymer network has begun to open, the solute particles would encounter regions of high local density where water penetration is still incomplete, resulting in GAL lodging within semi-hydrated aggregates. Such premature entrapment not only produces non-uniform solute distribution but also can lead to delayed or incomplete dissolution of GAL, ultimately affecting the microstructural uniformity and functional performance of the final hydrogel.

By contrast, allowing the polymer to undergo a controlled pre-hydration rest phase ensures that the HPMC chains possess the flexibility, spacing, and solvated surface characteristics needed to accommodate incoming GAL molecules through a combination of hydrogen bonding, hydrophobic interactions, and chain interpenetration mechanisms. The synergistic relationship between this brief rest period and the subsequent solute incorporation step produces a dispersion in which GAL molecules become fully integrated into the evolving three-dimensional polymeric network rather than adhering to partially hydrated particulates. As a result, the hydrogel precursor achieves a high degree of compositional homogeneity, setting the foundation for a structurally uniform Galangin-loaded hydrogel upon completion of the cold-swelling and network stabilization stages.

In an embodiment, the magnetic stirring performed by the LMS-1003 apparatus is executed using a PTFE-coated magnetic stir bar configured with an aspect ratio between 3:1 and 4:1, the geometry of the stir bar being selected to generate elongated vortical flow paths along the longitudinal axis of the mixing vessel such that the hydrogel precursor experiences periodic shear cycling that enhances homogeneity of GAL distribution within the matrix prior to cold storage, and wherein the distilled water used for preparing the hydrogel precursor is characterized by electrical conductivity not exceeding 2 μS/cm, and wherein the water is filtered prior to use through a 0.22 μm membrane filter to remove particulate contaminants that would otherwise act as nucleation points for localized HPMC aggregation or GAL precipitation during the 10-minute shear mixing stage.

In one embodiment, the magnetic stirring conducted using the LMS-1003 stirring apparatus employs a PTFE-coated magnetic stir bar having an aspect ratio between 3:1 and 4:1, a geometric configuration chosen specifically to produce an elongated vortical flow path extending along the longitudinal axis of the cylindrical mixing vessel. The increased length of the stir bar relative to its diameter enables the creation of a narrower, deeper central vortex and a correspondingly broader axial circulation pattern, which together generate periodic shear cycling across the entire fluid column. This flow behavior ensures that the hydrogel precursor, comprising the hydrating HPMC polymer and gradually introduced Galangin (GAL), experiences alternating regions of high and low shear stress, thereby promoting repeated solute redistribution and preventing localized accumulation of GAL or incomplete hydration of HPMC chains. The PTFE coating provides a chemically inert, low-friction surface that avoids solute adsorption and minimizes turbulence spikes, ensuring that hydrodynamic forces remain consistent throughout the 10-minute mixing period. For example, when a 35 mm×10 mm PTFE stir bar (3.5:1 aspect ratio) is used at 450 rpm in a 250 mL vessel, the resulting vortex depth and axial circulation patterns have been shown to eliminate microzones of GAL saturation, as confirmed by uniform turbidity readings across the mixing height.

The distilled water used to prepare the hydrogel precursor is limited to an electrical conductivity not exceeding 2 μS/cm, a threshold selected to ensure that the aqueous medium is free of ionic contaminants that could otherwise interfere with polymer chain hydration or alter GAL dissolution kinetics. Higher ionic conductivities, even at modest levels above this threshold, can influence the hydration shell structure around the HPMC chains, potentially accelerating or slowing dissolution rates in unpredictable ways and thereby compromising the reproducibility of the hydrogel consistency. Additionally, the distilled water is passed through a 0.22 μm membrane filter to remove submicron particulates, including silica residues, dust, and microscopic organic debris. These particles would otherwise act as heterogeneous nucleation points during mixing, causing localized HPMC aggregation or premature precipitation of GAL when exposed to shear gradients. Such nucleation-induced irregularities can lead to gel microstructural defects, including clusters of poorly hydrated HPMC or GAL-rich microdomains, which ultimately compromise the mechanical uniformity and diffusion behavior of the final hydrogel.

By combining the elongated vortical flow produced by the high-aspect-ratio PTFE-coated stir bar with the use of ultra-pure, low-conductivity, 0.22 μm-filtered water, this embodiment achieves a synergistic effect where mechanical and chemical purity conditions reinforce one another. The mechanically optimized flow field enhances uniform dispersion, while the chemically controlled aqueous environment eliminates extraneous nucleation surfaces and ionic perturbations. Together, these conditions ensure that GAL is dissolved consistently and incorporated evenly within the hydrating HPMC network during the critical 10-minute mixing stage. This coordinated interplay between hydrodynamic engineering and fluid purity enables the formation of a precursor mixture with superior homogeneity and structural predictability, directly translating to improved performance and stability in the final GAL-loaded hydrogel obtained after cold swelling.

In an embodiment, during the two-day cold-swelling period at 4° C., the sealed container is positioned such that the hydrogel precursor occupies between 40% and 70% of the container volume, thereby creating a defined headspace above the hydrogel mixture, the headspace functioning to stabilize hydrogel development by enabling pressure equilibration while preventing any compressive resistance that might alter the hydration patterns of the HPMC molecular chains during the slow-temperature swelling progression, and wherein the sealed vessel is placed upon a vibration-dampening platform during the two-day storage period, the platform comprising a viscoelastic composite capable of attenuating oscillatory energy above 5 Hz, thereby ensuring that the hydrogel precursor remains undisturbed in a static spatial configuration essential for the uniform inward diffusion of water molecules into the polymer microdomains of the HPMC matrix.

In an embodiment, during the two-day cold-swelling period at 4° C., the sealed hydrogel container is oriented such that the hydrogel precursor occupies between 40% and 70% of the vessel's total internal volume, a volumetric range selected to create a controlled and sufficiently capacious headspace above the precursor mixture. This defined headspace performs a critical thermodynamic and mechanical function by allowing internal pressure equilibration as the temperature of the mixture decreases from ambient to 4° C. As the aqueous HPMC-GAL system cools, both the liquid phase and the polymer matrix undergo slight volumetric contraction, and the presence of an unoccupied upper volume provides the necessary buffer to accommodate these changes without inducing mechanical compression of the hydrating polymer chains. If the vessel were filled beyond 70% capacity, the reduction in thermal volume could generate upward pressure on the parafilm seal or impose lateral compressive forces on the gel boundary, distorting the hydration front and potentially promoting anisotropic swelling. Conversely, filling below 40% would reduce the mass of polymer available for stable gel formation and could lead to exaggerated surface-air interactions, influencing the hydration equilibrium. Thus, maintaining the precursor within the defined volumetric window ensures a stable, pressure-balanced environment in which the HPMC chains can expand uniformly as water diffuses slowly into their microdomains during the low-temperature swelling process.

To further ensure that the hydrogel precursor experiences an uninterrupted, diffusion-dominated hydration pathway, the sealed vessel is placed upon a vibration-dampening platform for the entirety of the two-day storage period. The platform comprises a viscoelastic composite designed to attenuate oscillatory energy above 5 Hz, effectively neutralizing mechanical disturbances transmitted through the laboratory bench, refrigeration system, or ambient environment. Vibrational energy above this threshold is of particular concern because it can induce micro-mixing currents or slight shifts in the position of partially swollen polymer aggregates, thereby disrupting the delicate process of inter-chain hydrogen bonding and configurational stabilization that occurs during cold hydration. Even subtle oscillations can alter the spatial uniformity of water ingress into the HPMC matrix, creating regions of accelerated swelling or localized densification, which ultimately compromise the micro-porous architecture of the final hydrogel.

By ensuring that the vessel remains undisturbed in a fixed spatial configuration, the system favors inward diffusion of water molecules from the bulk phase into the amorphous polymer microdomains—a process essential for generating a structurally consistent, mechanically stable hydrogel network. This combination of controlled headspace management and vibration isolation enables slow, symmetrical hydration and swelling of HPMC chains, promotes uniform GAL entrapment within the forming polymer lattice, and establishes reproducible microstructural characteristics across batches. The synergy between volumetric headspace design and dynamic isolation thus ensures that the cold-swollen hydrogel develops with optimal physical homogeneity and diffusion properties.

In an embodiment, the final hydrogel mixture is subjected to visual inspection using a diffuse white-light source of 5600 K color temperature immediately following removal from cold storage, the inspection being performed while the parafilm seal remains intact, such that the operator evaluates internal phase uniformity through optical transmission and scattering characteristics without exposing the hydrogel matrix to ambient humidity exchange, and wherein the environmental conditions during the entire preparation procedure are controlled such that ambient relative humidity remains between 30% and 55%, and wherein no airflow exceeding 0.15 m/s is permitted across the open container during polymer hydration and GAL mixing steps, thereby preventing premature surface skin formation on the hydrogel precursor and ensuring consistent solvent evaporation rates during the active mixing interval.

In one embodiment, immediately after the two-day cold-swelling period at 4° C., the final hydrogel mixture is evaluated through a visual inspection step that employs a diffuse white-light source calibrated to a color temperature of approximately 5600 K, a specification chosen because it provides a neutral spectral profile that minimizes wavelength-dependent distortions in optical transmission and scattering patterns within the hydrogel. This inspection is intentionally performed while the parafilm seal remains intact, allowing the operator to assess internal phase uniformity without exposing the hydrogel surface to ambient humidity exchange or evaporative perturbations. The sealed-state inspection allows the observer to detect optical indicators such as uniform light scatter, absence of phase-separated streaks, lack of microbubble clusters, and a consistent translucency profile, all of which reflect homogenous polymer chain hydration and even GAL distribution across the hydrogel matrix. Removing the parafilm prior to inspection would risk introducing moisture gradients or surface contraction effects, which could obscure the true internal optical characteristics and lead to misinterpretation of the hydration quality.

To maintain reproducibility and prevent environmental interference during all preparation stages leading up to inspection, the laboratory conditions are tightly controlled such that ambient relative humidity consistently remains between 30% and 55%. This humidity range is selected to prevent excessive evaporation during mixing-which would artificially increase local polymer concentration—and to avoid hygroscopic absorption that would occur at higher humidity levels, potentially altering the rheology and hydration kinetics of the HPMC dispersion. In addition, the procedure requires that airflow across the open vessel during polymer hydration and GAL mixing be restricted to below 0.15 m/s, ensuring that no convective currents or localized drying occur at the surface of the precursor solution. Even mild air movement has been observed to trigger premature surface skin formation on hydrophilic polymer dispersions, particularly those containing HPMC, which forms thin dehydrated films when exposed to moving air. Such surface skins interfere with solute incorporation by creating diffusion barriers that prevent uniform GAL migration into the bulk and cause inconsistencies in gelation behavior during the subsequent cold-swelling period.

By maintaining low airflow and controlled humidity, evaporation is minimized during the active mixing interval, allowing solvent volume and viscosity to remain constant and ensuring that the hydrogel precursor maintains uniform hydration potential across the entire fluid column. When combined with the sealed optical inspection performed under standardized lighting conditions, the embodiment creates a synergistic operational environment where both the formation and evaluation of the hydrogel are shielded from external perturbations. This deliberate integration of environmental control and optical assessment guarantees that the hydrogel's structural uniformity—an essential quality parameter for GAL-loaded matrices—is accurately preserved, quantified, and validated without introducing artifacts or inconsistencies arising from uncontrolled ambient conditions. As a result, the method produces a hydrogel with reliable microstructural integrity, predictable performance characteristics, and reproducible GAL distribution-outcomes that depend critically on the tightly harmonized physical conditions described in this embodiment.

In an embodiment, the hydrogel precursor is mixed within a vessel comprising cylindrical walls having a diameter-to-height ratio between 1:1 and 1:1.6, the vessel geometry being chosen to generate a stable, centrally aligned vortex during shear mixing, thereby ensuring consistent distribution of kinetic energy across the depth of the solution, uniform dissolution of GAL throughout the polymer matrix, and symmetric hydration of HPMC chains prior to the cold-swelling phase, and wherein the gradual incorporation of Galangin (GAL) into the HPMC dispersion further comprises maintaining the centerline temperature of the dispersion within ±0.5° C. of ambient temperature by periodically monitoring the thermal profile with a Class A glass laboratory thermometer, and wherein the temperature stabilization is performed to prevent thermally induced viscosity fluctuations within the polymeric medium, such that the GAL molecules experience a consistent microviscosity environment during solvation and interpenetration into the hydrating HPMC chain network throughout the entire 10-minute mixing interval.

In an embodiment, the hydrogel precursor is mixed within a vessel whose cylindrical walls possess a diameter-to-height ratio between 1:1 and 1:1.6, a geometric configuration intentionally selected to promote the formation of a stable, centrally aligned vortex during the shear-mixing stage. This specific ratio ensures that the vortex generated by the rotating magnetic stir bar neither collapses prematurely against the vessel base nor elongates excessively along the vertical axis, both of which could lead to uneven shear distribution. Instead, the chosen geometry produces a well-defined, symmetric vortex column that transmits kinetic energy uniformly across the depth and breadth of the solution. Such homogeneity in hydrodynamic forces is essential for ensuring that Galangin (GAL) dissolves consistently throughout the HPMC dispersion, as the vortex entrains newly added GAL particles into the high-shear central region where rapid wetting and solvation occur before the fluid transports them outward toward lower-shear zones for complete integration. Additionally, the uniformity of mixing enabled by the vessel geometry supports symmetric hydration of the HPMC polymer chains, preventing localized over-hydration or under-hydration regions that could compromise the structural regularity of the hydrogel precursor before cold swelling.

During the gradual incorporation of GAL into the vortex-driven dispersion, the thermal state of the polymer-solute mixture is carefully maintained within ±0.5° C. of ambient temperature. This is achieved by periodically measuring the centerline temperature of the dispersion using a Class A glass laboratory thermometer, which provides high-precision thermal readings with minimal lag and without introducing extraneous heat exchange artifacts. Temperature control is indispensable because HPMC exhibits temperature-dependent viscosity, with even small deviations altering its pseudo-plastic flow behavior and affecting the rate at which GAL molecules can migrate, dissolve, and interpenetrate the hydrating polymer chain network. If the temperature were to drift below the defined tolerance band, the polymer matrix would thicken, slowing solute diffusion and creating inconsistent dissolution kinetics. Conversely, a slight elevation in temperature would reduce viscosity enough to destabilize shear uniformity, allowing some GAL particles to dissolve too rapidly while others remain insufficiently solvated.

By preserving a narrow thermal window and preventing thermally induced viscosity fluctuations, the embodiment ensures that GAL experiences a consistent microviscosity environment throughout the entire 10-minute mixing interval. This controlled rheological state promotes orderly solvation of the polyphenolic solute, facilitates its intermolecular interactions with the partially hydrated cellulose ether chains, and enhances uniform entrapment within the polymer network. The synergy between optimized vessel geometry and precise thermal stabilization thus produces a hydrogel precursor of high structural and compositional uniformity, enabling predictable gelation behavior during subsequent cold-swelling at 4° C. and ensuring reproducibility across multiple preparation batches.

In an embodiment, the hydrogel precursor is mixed in a vessel that has undergone a pre-conditioning step comprising rinsing with 70% ethanol, drying under laminar airflow, and subsequently equilibrating the vessel for at least 10 minutes to eliminate volatile residues, and wherein said pre-conditioning step is performed to ensure that the internal surface chemistry of the vessel remains inert with respect to GAL adsorption or polymer-surface interactions that may otherwise disrupt uniform matrix formation during HPMC hydration.

In an embodiment, the hydrogel precursor is mixed in a vessel that has undergone a pre-conditioning protocol designed to ensure chemical inertness and surface cleanliness prior to contact with the HPMC-GAL formulation. The pre-conditioning begins with rinsing the interior surfaces of the vessel using 70% ethanol, a concentration selected for its proven efficacy in dissolving organic residues, dislodging particulate contaminants, and disrupting microbial cell membranes without leaving behind hydrophobic or polymer-reactive films. The ethanol rinse removes adsorbed impurities that could otherwise interact with the polyphenolic structure of GAL or with the hydrophilic substituents of the HPMC chains during the critical hydration stage. After the ethanol wash, the vessel is dried under a laminar airflow cabinet, where the directional, particle-filtered airflow accelerates evaporation of residual solvent while preventing airborne contaminants from re-settling on the interior walls. This drying step also ensures that no droplets or moisture pockets remain that might alter the water content of the hydrogel precursor or initiate premature polymer swelling on localized regions of the vessel surface.

Following ethanol drying, the vessel is equilibrated for at least 10 minutes at ambient laboratory conditions to allow any trace volatile residues—including low levels of ethanol vapor—to fully dissipate. This equilibration step is essential because even minute quantities of residual ethanol can influence the microenvironment during early-phase HPMC hydration. Ethanol, being partially miscible with water and capable of disrupting hydrogen bonding, can transiently alter the hydration kinetics of cellulose ether chains or interfere with the dissolution profile of GAL. Allowing the vessel to equilibrate ensures that its surface returns to a neutral, non-reactive state consistent with the physicochemical expectations of borosilicate glass, thereby eliminating unintended solvent-polymer interactions.

The cumulative effect of this pre-conditioning procedure is that the internal surface chemistry of the vessel remains inert during the mixing and hydration stages. This inertness is critical because non-preconditioned surfaces can promote unwanted adsorption of GAL, especially given GAL's affinity for hydrophobic and partially polar surfaces due to its conjugated aromatic structure. Adsorption would result in solute losses, concentration gradients, or localized supersaturation zones that disrupt homogeneous distribution within the polymer matrix. Similarly, surface irregularities or chemical residues could initiate heterogeneous nucleation points where HPMC begins to swell unevenly or adhere to the vessel wall, generating non-uniform hydration fronts and inconsistencies in microstructural gel formation.

Through this controlled sequence of rinsing, drying, and equilibration, the vessel is rendered chemically and physically neutral, creating an environment in which the HPMC chains can hydrate uniformly and GAL can disperse and dissolve without interference from vessel-induced effects. This ensures reproducible formation of a stable and homogeneous hydrogel precursor, thereby supporting the structural integrity and therapeutic consistency of the final Galangin-loaded hydrogel formulation.

In an embodiment, the stirring operation is carried out at a rotational speed selected such that the vortex depth remains between 20% and 35% of the liquid column height, the vortex geometry enabling partitioning of the fluid into a high-shear central zone and a low-shear peripheral zone, and wherein GAL particles introduced into the vortex are entrained into the high-shear region for rapid dissolution before being advected toward the low-shear region, thereby producing cyclical solute redistribution within the hydrating HPMC matrix during mixing, and wherein the two-day cold storage at 4° C. is conducted inside a refrigeration system equipped with forced-air circulation restricted to an airflow velocity below 0.05 m/s at the shelf level, and wherein the hydrogel container is positioned at least 5 cm away from the refrigeration air outlets, thereby ensuring uniform cooling of the hydrogel precursor and preventing localized convective cooling patterns from influencing the polymer chain hydration symmetry across the entire bulk volume.

In an embodiment, the stirring operation is performed at a rotational speed calibrated such that the vortex formed within the mixing vessel achieves a depth corresponding to 20% to 35% of the total liquid column height. Maintaining the vortex within this depth range is essential because it creates a hydrodynamic environment that naturally partitions the fluid into two distinct shear regimes: a high-shear central zone concentrated along the vortex axis and a surrounding low-shear peripheral zone distributed near the vessel walls. When GAL particles are introduced into the system, they are immediately captured by the descending central vortex column and conveyed into the high-shear region, where intense fluid velocities and shear stresses rapidly disrupt particle boundaries, enhance interfacial wetting, and accelerate dissolution. Once partially solubilized, the GAL particles or microdroplets are advected outward and upward toward the low-shear peripheral zone, where shear thinning of the HPMC matrix promotes a slower, stabilizing dispersion phase. This cyclical transport of solute between high- and low-shear domains leads to continual redistribution of GAL throughout the hydrating polymer network, resulting in spatially homogeneous solute incorporation and preventing the formation of concentration gradients or undissolved agglomerates. The synergy between vortex depth control and shear zoning ensures that both polymer hydration and GAL solvation proceed in a coordinated manner, contributing to uniform gel microstructure and consistent functional loading.

Following mixing, the hydrogel precursor undergoes a two-day cold storage period at 4° C. inside a refrigeration system engineered to provide controlled, uniform cooling. The refrigeration chamber is selected such that its forced-air circulation is restricted to a low airflow velocity, remaining below 0.05 m/s at the shelf level. This low-flow condition is crucial because higher airflow velocities can generate localized convective cooling zones, causing differential temperature gradients around the vessel and potentially inducing uneven hydration or polymer contraction. By limiting the airflow, the hydrogel precursor experiences a stable and uniformly distributed cooling profile that supports symmetrical swelling of the HPMC chains as water continues to diffuse into the polymer microdomains during the cold-gelation stage. Additionally, the container is deliberately positioned at least 5 cm away from refrigeration air outlets to avoid direct impingement of cold air jets, which can create abrupt cooling fronts, freeze zones, or surface skinning phenomena that disrupt internal hydration uniformity.

Through this controlled placement and restricted airflow, the hydrogel precursor cools gradually and evenly, ensuring that the hydration-driven polymer restructuring occurs consistently across the entire bulk volume. The combination of precisely managed vortex geometry during mixing and carefully regulated thermal conditions during cold storage results in a hydrogel with uniform internal architecture, consistent Galangin distribution, and reproducible physicochemical characteristics essential for performance stability and batch-to-batch reliability.

In an embodiment, the container sealed with parafilm is placed in an upright orientation during the cold-swelling phase, the upright positioning ensuring a gravitationally stable hydrogel-air interface, and wherein the hydrogel precursor is allowed to undergo unidirectional vertical hydration expansion from bottom to top, such that hydration gradients remain minimal due to the absence of lateral fluid displacement forces that would otherwise arise if the container were tilted or horizontally oriented during the swelling period.

In an embodiment, the container sealed with parafilm is positioned in a strictly upright orientation throughout the entire cold-swelling phase at 4° C., the vertical alignment being essential to maintaining a gravitationally stable hydrogel-air interface. When the container remains upright, the interface between the hydrogel precursor and the headspace above it is not exposed to lateral slippage, surface skewing, or asymmetric stress distributions that could arise from even slight tilting of the vessel. The upright positioning ensures that the hydrogel precursor occupies the lower portion of the vessel in a uniform horizontal plane, allowing gravitational forces to act symmetrically across the polymer-solvent mixture. This stable interface prevents the formation of oblique hydration fronts and avoids the development of asymmetric gel layers that could compromise the structural integrity of the final hydrogel.

Under these conditions, the hydrogel precursor is permitted to undergo unidirectional vertical hydration expansion, progressing from the bottom of the vessel toward the top. This bottom-up hydration pattern naturally aligns with the direction of water influx into the partially swollen polymer domains, as water molecules migrate upward through the expanding HPMC matrix by diffusion while the matrix simultaneously elongates vertically. Such an arrangement minimizes hydration gradients within the bulk because the polymer chains hydrate and swell in a continuous, stratified manner without interference from lateral displacement forces. If the container were placed at an angle or in a horizontal orientation, gravitational redistribution of the semi-hydrated polymer could generate uneven fluid layering, causing the polymer chains to accumulate disproportionately toward one side of the vessel. This would lead to localized densification, asymmetric pore formation, and non-uniform GAL entrapment, ultimately altering diffusion characteristics and mechanical consistency of the final hydrogel.

By maintaining the vessel upright, the swelling occurs in a vertically coherent fashion, preserving uniformity in polymer chain entanglement, porosity development, and hydrogel architecture. The absence of lateral hydrostatic pressure gradients ensures that the hydration fronts advance at comparable rates across the entire horizontal cross-section of the vessel, preserving microstructural symmetry. This controlled orientation, combined with the static environment and parafilm sealing, therefore ensures a consistent cold-swelling process and contributes to the reproducibility and performance stability of the resulting Galangin-loaded hydrogel.

Skin wound repairing is a natural mechanism that comprises a series of intricate cellular and bimolecular pathways that recover injured to its original condition. The essential biochemical tissue restoration procedure is involved inflammation, migration and proliferation of cell, and final remodeling. Rapid wound repair outcomes from the ordered sequence of healing proceedings. In normal healthy subjects, the mechanism of tissue repair happens at an ideal degree, nevertheless in individuals with diabetes, it is typically postponed or compromised. In diabetic subjects, wound tissue ischemia, hypoxia, and hyperglycemia impede with the progression of these planned repair phases, leading to non-wound tissue repair or protracted wound repairing as well as a number of clinical problems. Prolonged or non-repairing of diabetic injuries represent a global medical challenge. Although diabetic skin injuries have partially improved as a result of contemporary treatments, the prevalence, amputation, and death rates of diabetic wounds are still high due to the complexity of the disease's pathophysiology and mechanism. Finding new or better medications that can both lower costs and successfully treat diabetic wounds is therefore urgently needed.

The present invention aims to discover the potential impacts of GAL and MEBO® ointment as a reference drug (each alone) against diabetic wound, hoping this natural compound (GAL) to enhance the diabetic wound healing process via mitigating the risk elements contribute in non-healing process, including, oxidative stress, inflammation, angiopathy, and collagen degradation.

The present investigation revealed that the skin injury recovery in rats with diabetes was postponed versus normal rats. The results also showed that the diabetic rats' wounds did not recover and the remained area of skin tissue injury on day 14 was greater than that of the nomal rats, indicating that the recovery rate of wound in hyperglycemic animals rats was slower than that of the normal counterparts. These findings align with prior experimental evidence showing delays in skin wound recovery in hyperglycemic experimental models. Evidence suggests that many risk factors take parts in postponing the recovery of diabetic skin tissue injury and augmented the vulnerability to infections, including poor glycemic control, peripheral neuronal damage, decreased blood supply due to endothelial damage, and immune-pathy. In addition, some authors suggested that the deferred wound recovery in diabetic rats may be related to extreme oxidative stress, continued inflammation, and compromised angiogenesis. Diabetic wounds can be advanced to serious consequences like skin tissue death, surgically removal of a lower-limb, and vulnerability to infections. This will impact the modality of life and healthcare facilitation. Topical administration of vehicle, or MEBO® ointment or GAL, significantly reduced the dimensions of wound skin compared with diabetic untreated rats, as shown by increases in percentages of wound healing progression on days 3, 7, 10, and 14. Treatment with GAL and separately MEBO® ointment was effective in wound healing by day 14. GAL-treated rats showed 95% closure of the wound area by day 14, indicating nearly complete closure of the wound area. This degree of healing exceeded that observed in all other groups, including the normal control (87.33%±6.25) and the MEBO® ointment-treated group (87.00%±9.29), which themselves demonstrated efficient physiological and pharmacological wound repair, respectively. The vehicle group exhibited a moderate effect (41.57%±9.32), while the untreated diabetic group showed significantly delayed healing, with a mean contraction of only 20.00%±7.40. The wound healing effect of GAL may suggest that this flavonoid may have modulating impacts on oxidative stress and inflammatory reaction, as well as angiopathy induced in response to skin damage. Similarly, the useful wound recovery influence of GAL was evaluated on L929 mouse fibroblast utilizing an in vitro scratch technique. The authors reported that administration of 25 and 50 μM provided nearly complete closure at the end of 36 hours. The GAL may potentially prevent hypertrophic scar formation. In addition, a former study revealed that MEBO® ointment considerably caused complete wound curing in hyperglycemic rodents 14 days post-treatment.

In the present invention, H&E histological functions and MTC staining techniques were pivotal in assessing the healing processes in diabetic wounds. H&E staining effectively highlights cellular structures, allowing for the evaluation of tissue architecture, inflammatory cell infiltration, and epithelialization status. This method is essential for visualizing the overall morphology of the wound site, wherein it is noted the different cellular responses in diabetic wounds. Conversely, MTC staining provides a clearer distinction of collagen fibers, enabling the assessment of collagen deposition and organization, which are crucial for wound healing. Studies have shown that MTC staining reveals the maturation of collagen fibers, indicating the progression of healing.

In the current study, the untreated diabetic wounds exhibited extensive inflammatory cell infiltration, a wide wound gap, disorganized granulation tissue lacking collagen fibers, and absent re-epithelialization, all indicating delayed healing. Equivalently observed in vehicle-treated wounds. These findings are consistent with previous research. Specifically, it was emphasized that diabetic wounds are characterized by prolonged inflammation and impaired re-epithelialization. Similarly, it is reported comparable histopathological changes, including the absence of an epithelial layer beneath the necrotic scab, further reflecting significant delays in the healing process.

In contrast, wounds treated with MEBO® in the positive control group showed significant improvements in histological outcomes. Enhanced surface re-epithelialization, organized collagen remodeling, and a reduction in inflammatory infiltrate were observed, aligning with findings from literature. Additionally, it was noted that MEBO® treatment improved collagen maturation and reduced the presence of inflammatory cells, indicating a more organized healing response. These results suggest that MEBO® may promote a structured healing process, facilitating the transition from the inflammatory phase to the proliferative phase of wound healing. Furthermore, studies involving flavonoids, such as quercetin and catechin, have reported similar positive effects on diabetic wound healing. For instance, it was found that Sinapic acid treatment significantly enhanced re-epithelialization and collagen deposition in diabetic rats. This body of evidence suggests that flavonoids can modulate inflammatory responses and promote collagen synthesis, contributing to improved healing outcomes. In the present invention, the application of GAL as a flavonoid resulted in remarkable histological improvements, with treated wounds exhibiting complete re-epithelialization and well-organized collagen deposition. This finding aligns with the observation from literature, wherein it is noted that GAL enhances tissue protection and reduces inflammation and pathological fibrosis in isoproterenol-induced cardiac injury in Wistar rats. Specifically, the study demonstrated that GAL pretreatment markedly reduced myocardial fiber degeneration and inflammatory cell infiltration, while also preventing excessive collagen accumulation and preserving normal tissue architecture.

According to reports, one of the key processes in the pathophysiology of diabetic wounds is oxidative stress. The term “oxidative stress” describes a number of harmful processes brought on by an imbalance between the inadequate antioxidant defenses and the excessive production of ROS. This inequity seems as a common risk element in several disease conditions where oxidative harm results in tissue destruction due to cell necrotic death.

This invention revealed that a noticeable upsurge in the immunostaining expression of MDA (an indicator of oxidative stress and membrane lipid peroxidation) concomitant with significant depletion in the expression of antioxidant defense markers, namely Nrf2, NQO1, SOD and CAT in wounded tissue of diabetic rats compared to control non-diabetic ones. The current results are in accordance with the data obtained from another invention, which reported that remarkable increase in MDA accompanied by decreases in the antioxidant enzymes. The elevation of tissue MDA may be ascribed to the reaction of free radicals with cell membrane lipids to form MDA as a final product of lipid peroxidation, leading to tissue damage. Lipid peroxidation is considered the chief biochemical process causing oxidative impairment to cell organization and cell death. Generation of MDA can suppress protein anabolism, enzymatic reactions and immune cells communication. MDA, is a genotoxic elements can form adducts with DNA, leading to cytotoxicity, genotoxicity, and carcinogenicity. At the same time, the depletion of SOD and CAT blocks the enzymatic protection versus superoxide radicals and H₂O₂ facilitated injury. SODs lower the quantity of superoxide anion free radical (O-2), which harms cells at high levels, and catalyse its dismutation into O2 and H2O2. CAT breaks down hydrogen peroxide, preventing the formation of highly reactive hydroxyl radicals that could harm cells. NQO1 is a ubiquitous intracellular detoxifying enzyme that mediated the conversion of reactive quinones to their less reactive and less toxic hydroquinone forms. The imbalance between ROS levels and antioxidant enzymes in diabetic wounds leads to ROS accumulation in endothelial cells, which mediates endothelial dysfunction, neuropathy, or local infection, resulting in delayed repairing or non-repairing of diabetic skin injury. The current drop SOD, CAT and NQO1 in wounded tissue of diabetic rats may attribute to deactivation brought on by an excess of free radicals generation and/or inactivation of Nrf2. Nrf2 is a transcription element that has a vital part in regulating the generation of antioxidant molecules as well as numerous genes linked to redox balance. Nrf2 can counteract disadvantages, including oxidative and inflammatory cellular damage. Nrf2 exerts a defending impacts on several diabetic difficulties. Thus, the current inactivation of Nrf2 may have a role in the depletion of SOD, CAT and NQO1 via affecting their expression in wounded skin of diabetic rats and this may aggravate the generation of free radical and postponed diabetic wound recovery. It has been reported that the diabetic rodents with Nrf2 shortage show a late wound curative rate, representing the vital function of Nrf2 in repairing diabetic skin injury. Thus, activating Nrf2 can accelerate the rate of skin damage recovery in diabetic conditions.

Topical administration of vehicle, or MEBO® ointment or GAL pointedly prohibit the tissue creation of MDA and boosted the manufacture of Nrf2, SOD, CAT and NQO1 in diabetic wound tissue compared to untreated diabetic rats. GAL proved to be the most effective agent in modulating all these parameters to the normal expression level. Similarly, previous inventions have shown that GAL can reduce MDA levels in models of acetaminophen-induced liver injury and cardiometabolic disorders. Furthermore, it was stated that GAL heightened the antioxidants enzyme functions in cortex and hippocampus of an experimental cerebral ischemia model. This beneficial influence of GAL in contradiction of oxidative stress in the wounded tissue of diabetic rodents may be attributed to the strong anti-oxidative stress and antioxidant abilities of this compound. The enhancing effect of this flavonoid on the expression of Nrf2 presented in the current invention may have the main role in the manufacturing of enzymatic antioxidants. Renovating of Nrf2 manufacturing by GAL can be recommended as a strong curative approach in diabetic wounded skin recovery via alleviating oxidation tissue injury. It was reported that GAL improves skin inflammatory damage in mice with psoriasis via stimulation of the Nrf2/HO-1 signal mechanism. Besides, GAL can neutralize free radicals by releasing hydrogen atoms from its hydroxyl group. Flavonoids help mitigate oxidative damage by hunting reactive elements providing defence against a range of illnesses. The antioxidant effect of MEBO® ointment may be attributed to the ability of sesame oil present in MEBO® to scavenge free radicals due to its antioxidant properties.

Inflammation is another important element in the onset of diabetic wounds. This work demonstrated that pronounced elevation in the concentrations of histochemical immunostaining expression of inflammatory elements (TLR4, IL-6, IL-1β, TNF-α, and Cox2) in the injured skin of diabetic rats with respect to normal ones. Similarly, previous publications stated that the generation of inflammatory proteins such as IL-1β, IL-6, TNF-α, and Cox2, was raised in diabetic wound tissue. In current invention, the impaired wound repairing in hyperglycemic rodents may be related to the abnormalities of inflammatory risk proteins as one of the risk elements in delaying diabetic wound healing. Suitable expression of pro-inflammatory mediators is essential to recruit immune cells and eradicating any pathogen from the damaged site. However, extended production of these mediators is unfavourable, as it can cause the development of persistent injuries. An invention has demonstrated that the disruption of upstream receptors that start inflammatory reactions, such as the toll-like receptor (TLR) family, is considered a key factor in the worsen of diabetic wounds. The upsurge in TLR4 generation during hyperglycemia triggering the production and activation of NFκB, and inflammatory mediators, such as IL-6, TNF-α, and Il-1β, causing a decline in wound repairing. Several of these signaling proteins aggravate the inflammatory profile by encouraging the production of more pro-inflammatory intermediaries. Furthermore, TNF-α triggers NF-κB activation, which raises the release of additional inflammatory intermediates and COX-2 over-generation, which contributes significantly to tissue inflammation by catalysing the metabolic conversion of arachidonic acid into prostaglandins.

Topical treatment of diabetic rats with vehicle, MEBO® ointment, or GAL markedly reduced the levels of the inflammatory markers (TLR4, IL-6, IL-1β, TNF-α, and Cox2) concerning diabetic untreated rats. GAL was a useful agent in restoring all the inflammatory markers close to normal expression, indicating its potential anti-inflammatory beneficial activity. GAL showed anti-inflammatory useful influence on many ailment experimental examples, both in vivo and in vitro, including foot illnesses. GAL modulates a number of signalling mechanisms that contribute to the synthesis of pro- and anti-inflammatory intermediates. Thus relieving inflammation. GAL can prevent NF-κB stimulation, so impeding its ability to recruit the chain of inflammatory responses. Accordingly, GAL-induced suppression of NF-κB can diminish the concentrations of many pro-inflammatory molecules, including TNF-α, IL-1β, and IL-6. Additionally, GAL can inhibit the inflammation via suppressing the manufacture of COX-2, which is accountable for the creation of inflammatory prostaglandin E2 (PGE2). Moreover, GAL modulates the generation of Toll-like receptor 4 (TLR4). Manipulating cellular communication cascades through receptor targets like TLR-4 may provide a novel strategy for trying to promote healing in diabetic ulcers, by direct reduction of inflammation. In vivo inventions have demonstrated that systemically blocking TLR-4 can heal diabetic skin damage and lessen pro-inflammatory characteristics. The anti-inflammatory impact of MEBO® may attribute to its active compound, β-sitosterol, which has been shown to have anti-inflammatory useful impact.

Another important component in the underlying cause of diabetic skin injury is angiopathy, or vascular destruction. The current results revealed depletion in the expression of angiogenesis indices, namely, VEGF-A, TGF-β, and Platelet-derived growth factor receptor A (PDGFR) in skin injured tissue of diabetic rodents versus normal ones. The present results are in harmony with other inventions that reported reduced VEGF, TGF-β, and PDGFR secretion from diabetic wound tissue. From the current data, it can be mentioned that the reduction in these growth elements may affect the mechanism of neovascularization in wound tissue, causing a postponement in wound repaire. This suggestion is supported by the statement that late wound recovery is caused by a reduction in some growth factors in skin tissue. Endothelial cell quality and neovascularization play a vital role in wound healing. Vascular endothelial cells modulate the quantities of vasoactive mediators like ENOS to control vessels constriction and expansion. In repairing of skin damage, VEGF primarily regulates endothelial cells at various angiogenesis phases. In the inflammatory step, this growth element upsurges vascular permeability, influences endothelial cell production of selectin and intercellular adhesion molecules, and facilitate leukocyte attraction to the damaged tissue. In the proliferation phase, VEGF potentially facilitate the immigration and multiplication of endothelial cells, In contrast, it promotes the creation of vascular lumens during the remodelling stage by inducing endothelial cell assembly. According to in vivo invention, the endothelial cells of arteries in hyperglycaemic setting lose their function. They are more likely to undergo apoptosis, removed, and enter the bloodstream, which can result in angiogenesis ailments. Prolonged hyperglycaemia induces endothelial cells deterioration and malfunction and compromised angiogenesis. Hyperglycemia harms blood vessels, which results in decreased NO production and increased atherosclerosis, accumulation of platelets, and a contraction of blood vessels. Diabetic subjects frequently have peripheral artery ailments, which reduces the flow of blood to the limbs, resulting in ischaemia and postponing the repairing of skin damage. The following routes are mostly responsible for this damage: (a) polyol pathway, (b) elevated levels of AGEs, (c) RAGE over-generation, (d) stimulation of different protein kinase C variants, and (e) excessive induction of hexosamine mechanism. In hyperglycemia, the suppression of NOS due to peripheral neural damage and arterial illness causes a falling in blood flow by vessel contraction. The absence of endothelial progenitor cells (EPCs) in the injured tissue impedes the creation of new capillaries and prolonged the rate of wound recovery.

The TGF-β plays pivotal roles in both tissue development and repair, and is abundantly released from platelets immediately following injury. It contributes in skin damage recovery by guiding the immune cells to the damage location throughout the inflammatory period, supporting the development of granulation tissue and ECM buildup throughout the proliferative period, and swiftly converting collagen type III to type I during the remodeling step. Additionally, by boosting the transcription of the genetic factor for collagen, proteoglycans, and fibronectin, TGF-β upsurges the manufacture of matrix elements. Notably, it additionally blocks the release of proteases that break down matrix, while simultaneously stimulating the synthesis of tissue inhibitors of metalloproteinases (TIMPs), thereby preserving matrix integrity. In addition, TGF-β facilitates resurfacing of damaged skin with new cells by helping keratinocyte immigration via the triggering of forkhead box-1 (Fox-1) next receptor binding at the cell. Likewise, it has been implicated in angiogenesis, granulation tissue remodeling, and scar development, which are earlier phases of wound recovery. Accordingly, the observed downregulation of TGF-β production in the wounded skin of diabetic rodents in the present invention may represent a critical factor contributing to impaired healing. This finding aligns with clinical observations reporting reduced TGF-01 manufacture in biopsy specimens from diabetic subjects with skin sores, which is closely linked with prolonged wound repairing.

PDGFRA has important functions in angiogenesis. By binding to its ligand (PDGF), PDGFRA can participate in the wound healing process. Increased PDGF/PDGFR signaling has been demonstrated to support microvascular regrowth, chemo-attraction of immune cells to the injured region, and fibroblast replication and multiplication. In hyperglycemic experimental animals, the invention reported that PDGF/PDGFR signaling helps damaged tissue repairing by boosting the creation of new connective tissue and blood capillaries at the site of injury. PDGF/PDGFR signaling can influence the mechanism of skin injury repairing via management of inflammation, proliferation, and remodeling. Thrombocytes and phagocytic immune cells congregate at the injured site and secrete a variety of growth elements (such as PDGF, TGF-β, and IL-6) that contribute in the recovery stages of damaged skin. In the inflammatory step, PDGF/PDGFR signaling facilitates the chemo-attraction of neutrophils and macrophages in the injured tissue. During the inflammatory period, macrophages can eliminate pathogenic bacterial in the wound via generating. In hyperglycemic animals, PDGF/PDGFR signalling has been shown to raise fibroblasts and microvascular during the proliferation period, repairing skin lesion. In remodeling step, granulation tissue is substituted by scarring tissue, and both collagen anabolism and catabolism happen throughout this period. PDGF/PDGFR signaling contributes in wound remodeling by helping the release of TGF2-B1 by fibroblasts, boosting the anabolism of ECM components including collagen in the extracellular matrix, and facilitating skin remodeling in the last step of recovery. Thus, the present suppression of PDGFR in diabetic rat wound tissue may affect PDGF/PDGFR signaling, consequently reducing diabetic wound healing.

According to the above-mentioned findings, VEGF, TGF-β and PDGF/PDGFR signaling pathways are essential for different mechanisms of wound recovery. Decreasing the production of angiogenic elements greatly slows down the curative process of damaged tissue. Thus, keeping the balance of vascular endothelial cells is essential for the treatment diabetic skin injury. Therapeutic efforts targeting these angiogenic factors may be useful in the treatment of skin tissue injury. Topical treatment of vehicle, MEBO® ointment, or GAL to wounded tissue of diabetic rats, significantly induced the production of the angiogenic parameters (VEGF, TGF-β, and PDGFR) versus diabetic untreated rats. Inducing such growth factors in diabetic wounds by GAL or MEBO® ointment may have a potential role in promoting collagen synthesis, re-epithelialization, and keratinocyte migration, which may explain their accelerating impact in repairing damaged skin of diabetic rat presented in the current invention. This finding may highlight the therapeutic potential of both GAL and MEBO® ointment in treating diabetic wounded tissue. To the best of our information, this invention shows for the initial time how GAL affects VEGF, TGF-β, and PDGFR production in diabetic skin injury. The therapeutic impact of MEBO® on tissue damage repairing of cutaneous excisional wounds in experimental animals was investigated. According to the findings, applying MEBO® greatly increased VEGF generation, improved the growth of granulation tissues in cutaneous injuries, accelerated injury closure, increased fibroblast numbers and generated new blood vessels. Additionally, it was revealed that administration of MEBO® to the wounded skin of diabetic rats can quickly create granulation tissue, reconstruct collagen in damaged skin, and enhance re-epithelialization and migration ability of keratinocytes, proposing the strong influence of MEBO® in curing diabetic skin damage. MEBO® contains sesame oil, β-sitosterol with anti-inflammatory properties, and berberine, with antimicrobial activity.

In this invention, the data of qPCR demonstrated that considerable depletion in Col1A1 and Col4A1 synthesis in the wounded tissue of diabetic rodents with respect to the control non-diabetic counterparts. The result of this invention is coped with other findings suggesting that slower proliferation rate of fibroblast in addition to the alteration of their structure within the diabetic wound may relate to a diminution in the synthesis of ECM elements, including collagen, which could contribute to impaired wound healing. On the other hand, the present depletion in TGF-β and PDGFR levels may ascribe to a decrease in collagen expression. TGF-β suppressing the release of proteases that cause matrix breakdown and boosts creation of tissue inhibitor of TIMP, to inhibit the breakdown of the matrix. Thus, the decrease in the level of TGF-β in diabetic damaged skin can cause an imbalance of MMPs/TIMPs. This imbalance leads to further degradation of the ECM, angiogenesis inhibition, granulation tissue breakdown and deposited collagen. In the final steps of repair, PDGF/PDGFR signaling causes tissue reconstruction by encouraging the production and secretion of extracellular matrix (ECM) constituents, including collagen. Impairing of PDGF/PDGFR signaling in diabetic damaged tissue may cause a direct suppressing influence on collagen expression. Previous findings confirmed that hyperglycemia can impair fibroblast migration, deposition of the ECM, angiogenesis and the creation of granulation tissue during the proliferative phase of damaged tissue recovery via its direct effect on the interactions between diverse cell kinds and the ECM, which is consecutively remodeled and reconstructed during this process.

Treatment of wounded tissue of diabetic rats with vehicle, or MEBO® ointment or GAL significantly increased the generation of Col1A1 and Col4A1 in the wounded skin of diabetic rats with relation to untreated diabetic wounded tissue. This result may confirm the potential impact of the used agents on diabetic wound healing. This finding is supported by previous in vivo invention revealed that application of GAL different doses significantly improved skin photo-damage of C57BL/6J nude mice by encouraging TGFβ/Smad signaling for collagen creation. Additionally, the invention discovered that GAL inhibited the breakdown of collagen by activating the TGFβ/Smad pathway in HS68 cells and decreasing the quantity of H2O2-enhanced matrix metalloproteinase (MMP-1). Furthermore, an invention indicated that GAL possesses collagenase inhibitory effects in contradiction of photo-aging in dermal fibroblast. Moreover, it was declared that topical administration of MEBO® ointment markedly promoted the expression of collagen in the renewed skin of diabetic rodents, proposing that MEBO® therapy for diabetic skin damage aids in the repair of damaged skin.

The results from the present invention clearly indicates that applying GAL topically to the wounded skin of diabetic rats could greatly induce diabetic wound healing. GAL-treated rats exhibited the highest wound closure rate by day 14 and progressed more rapidly into the remodeling phase. GAL can expedite wound recovery by suppressing the oxidative stress index MDA, and improving the antioxidant activities of SOD, CAT, and immune-expression of antioxidant indices, namely Nrf2, and NQO1. The data also illustrated that GAL could suppress the wound inflammatory response in wounded skin through down-regulating the production of inflammatory indices, namely, IL-6, TNF-α, IL-1β, COX-2, and TLR4. At the same time, GAL could promote the angiogenesis pathways on the wound surface by increasing the generation of angiogenic elements, such as VEGF, TGF-β, and PDGFR. Moreover, GAL promotes collagen deposition by upregulation of Col1A1, and Col4A1 mRNA expression. Enhancing the production of such growth markers may have important roles in accelerating revascularization, improving blood flow on the wound surface, promoting collagen synthesis, and re-epithelialization. GAL was the effective agent in modulating most of these parameters close to the normal expression level. The present invention propose that using GAL in the treatment of wounded tissue may be considered a potential strategy in accelerating wound healing, especially diabetic ulcer wounds.

These findings from the present invention shows GAL as a promising therapeutic agent for accelerating diabetic wound healing. However, further research are necessary to fully elucidate the molecular mechanisms underlying GAL's effects, particularly its role in modulating oxidative stress, inflammatory pathways, and collagen synthesis. Investigating the intracellular signaling cascades such as NF-κB, MAPK, MMPs, and TGF-3/Smad pathways, may provide valuable insight into how GAL influences tissue regeneration. Additionally, future work should focus on optimizing the formulation and delivery method of GAL for topical applications, such as using nanotechnology-based systems. Developing GAL-loaded nanoparticles, nanogels, or liposomal formulations, and incorporating it into wound dressings or patches, may enhance its bioavailability at the wound site, stability, skin penetration, and thereby improving its therapeutic efficacy. Moreover, dose-response and time course studies are required to identify the optimal concentration and duration of GAL treatment. Establishing standardized protocols will support the development of practical therapeutic applications. Future studies should involve longer observation periods to assess the durability of the healing effects and the potential for scar formation or recurrence of wounds. Incorporating additional biomarkers of angiogenesis, tissue remodeling, and immune modulation would deepen understanding of the molecular mechanisms underlying GAL's activity. Finally, while the current results are promising in the diabetic rat model, future preclinical studies on larger animal models, followed by well controlled human clinical trials, are critical to validate GAL's efficacy and Safety. These steps are essential to translate experimental findings into a viable clinical solution for diabetic foot ulcers and related complications. Although comparative studies with existing wound healing agents could position GAL in the therapeutic landscape, either as a standalone option or as part of a combinatory approach.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A method for synthesizing a Galangin-loaded hydrogel formulation, the method comprising:
weighing 5 g of Galangin (GAL) and 1.5 g of hydroxypropyl methyl cellulose (HPMC) using an analytical balance comprising a RADWAG AS 110/X configured for ±0.1 mg resolution;
dispersing the 1.5 g of HPMC in 100 mL distilled water at room temperature while continuously agitating using a magnetic hotplate stirrer comprising an LMS-1003 device;
gradually introducing the 5 g of GAL into the stirred HPMC dispersion and maintaining shear mixing for 10 minutes;
sealing the mixture in a container with parafilm; and
storing the sealed mixture at 4° C. for two days to permit complete hydrogel swelling and structural stabilization, wherein the step of dispersing the hydroxypropyl methyl cellulose (HPMC) in distilled water comprises initiating polymer hydration by exposing the HPMC powder to a pre-established vortex of the aqueous medium, the vortex being created by the LMS-1003 stirrer operating at a rotational speed between 350 and 600 rpm, and wherein the polymer hydration is controlled such that the HPMC particles undergo sequential wetting, boundary-layer dissolution, and chain disentanglement, thereby generating a progressively thickening colloidal matrix in which the hydroxyl-substituted cellulose chains expand through hydrogen bonding with the aqueous phase while avoiding lump formation through maintenance of constant shear gradients across the depth of the vessel, and wherein the step of gradually dissolving the Galangin (GAL) into the hydrated HPMC matrix further comprises controlling the solute introduction rate such that each increment of GAL is added only upon reaching a predetermined optical turbidity threshold indicative of polymer-solute intercalation, wherein such intercalation involves non-covalent interaction between the polyphenolic structure of GAL and the ether-substituted anhydroglucose units of HPMC, and wherein the GAL introduction is carried out in aliquots of 0.25-0.50 g at intervals of 15-25 seconds to maintain uniform solute dispersion and prevent localized crystallization or agglomeration during the shear-induced solubilization phase.

2. The method of claim 1, wherein the shear mixing for 10 minutes comprises maintaining a controlled laminar-to-transitional flow regime within the hydrogel precursor solution, the flow regime being defined by a Reynolds number between 1800 and 2300 for the selected vessel geometry, such that the hydrodynamic forces generated by the stirrer enable the GAL molecules to undergo homogeneous distribution across the HPMC polymeric network while simultaneously promoting partial alignment of HPMC molecular chains, thereby facilitating uniform spatial encapsulation of GAL within the hydrogel structure prior to the cold-swelling step.

3. The method of claim 1, wherein the sealing step using parafilm comprises applying a multi-layered, stretch-sealed polymeric membrane around the opening of the container, the membrane being tensioned to generate an airtight closure capable of restricting vapor permeability to below 10 g·m⁻²·day⁻¹ at ambient pressure, thereby inhibiting moisture loss and preventing atmospheric carbon dioxide or environmental particulates from contacting the hydrogel precursor during the swelling period, and wherein the parafilm sealing further provides a static barrier that maintains the pressure equilibrium of the headspace above the hydrogel mixture.

4. The method of claim 1, wherein the storing of the sealed container at 4° C. for two days comprises maintaining the hydrogel precursor under a temperature-controlled environment in which the thermal gradient across the vessel does not exceed ±0.3° C., and wherein the reduced temperature induces contraction of the HPMC polymer chains followed by gradual re-expansion as water molecules migrate into amorphous polymer domains, thereby forming a three-dimensional gel network whose pore size distribution remains within a micro-scale range dictated by the degree of hydration achieved during the cold-swelling period, and wherein the distilled water used for HPMC dispersion is first subjected to a degassing step comprising vacuum extraction at <20 kPa for 5-10 minutes to remove dissolved gases that would otherwise cause microbubble retention within the hydrogel matrix during polymer swelling, and wherein the degassed water is subsequently equilibrated to room temperature prior to polymer hydration to ensure consistent diffusion coefficients for both water and GAL during dissolution.

5. The method of claim 1, wherein the GAL is introduced into the HPMC dispersion under illumination conditions restricted to <200 lux to prevent photochemical alteration of the GAL molecular structure, and wherein the GAL powder is pre-sieved using a stainless-steel mesh of 100-150 μm pore size to ensure granulometric homogeneity during incorporation into the polymer matrix, thereby ensuring consistent dissolution kinetics across all GAL particles during mixing, and wherein the container in which the GAL-HPMC mixture is sealed comprises a borosilicate glass vessel having a thermal expansion coefficient below 4×10⁻⁶/° C., the vessel being selected to ensure that no micro-structural deformation occurs during the two-day cold-swelling phase at 4° C., and wherein the interior surface roughness of the vessel is restricted to <0.4 μm Ra to prevent surface-induced aggregation of GAL or non-uniform HPMC hydration near the vessel walls.

6. The method of claim 1, wherein the hydrogel mixture is stored under static conditions, and wherein the static placement includes isolation from vibrational sources exceeding 0.1 g acceleration to prevent mechanical disruption of the hydration-driven network formation occurring within the HPMC matrix, such that the polymer chains undergo uninterrupted hydration, swelling, and entanglement as water molecules diffuse into the inter-chain regions to form the final hydrogel configuration, and wherein the HPMC used in the hydrogel formulation is selected to comprise a hydroxypropoxy substitution degree (DS) between 0.1 and 0.25 and a methoxy substitution degree (MS) between 1.1 and 1.7, and wherein these substitution levels govern the hydration behavior of the polymer during the two-day swelling step at 4° C. such that the resultant hydrogel exhibits a micro-porous network architecture with diffusion characteristics determined by the chemical modification pattern of the cellulose backbone.

7. The method of claim 1, wherein the step of mixing GAL into the HPMC dispersion further comprises controlling the pH of the aqueous medium within 6.0-7.0 to maintain GAL in a non-ionized state, and wherein the pH control is achieved through monitoring using a glass-electrode pH meter configured to ±0.01 pH accuracy without introduction of any additional buffering agents not disclosed in the formulation, and wherein following the two-day swelling period at 4° C., the hydrogel is equilibrated at room temperature for a period of 30-45 minutes while remaining sealed under parafilm, the equilibration step enabling thermal relaxation of polymer chain conformations without exposure to ambient humidity fluctuations that would otherwise affect the stability of the hydrated hydrogel network.

8. The method of claim 1, wherein the introducing of GAL into the hydrated HPMC dispersion further comprises performing continuous rotational shear mixing such that the shear field generated within the vessel produces both radial and axial flow components, wherein the stirrer's magnetic coupling induces alternating torque fluctuations that facilitate disruption of micro-aggregates of GAL, and wherein the GAL-containing dispersion is maintained at a temperature between 22° C. and 26° C. during the mixing interval to avoid viscosity shifts in the polymer matrix that would otherwise alter the solute mobility profile and impede uniform solvation of GAL's polyphenolic structure, and wherein the GAL added to the hydrogel precursor is pre-conditioned by storing the GAL in a desiccated chamber maintained below 10% relative humidity for at least 12 hours prior to weighing, such that the moisture content of the GAL powder remains below 0.5% w/w, thereby ensuring predictability of dissolution kinetics upon contact with the aqueous HPMC medium and preventing hydration-induced clumping during the staged solute incorporation step.

9. The method of claim 1, wherein the HPMC dispersion undergoes a pre-hydration rest phase lasting 2-4 minutes immediately after initial polymer wetting and before GAL is introduced, the pre-hydration phase permitting partial unraveling of the cellulose ether chains, such that the polymer viscosity profile transitions toward a pseudo-plastic state, thereby allowing the subsequently introduced GAL molecules to interdigitate with the expanding polymeric network rather than lodging within partially hydrated polymer aggregates.

10. The method of claim 1, wherein the magnetic stirring performed by the LMS-1003 apparatus is executed using a PTFE-coated magnetic stir bar configured with an aspect ratio between 3:1 and 4:1, the geometry of the stir bar being selected to generate elongated vortical flow paths along the longitudinal axis of the mixing vessel such that the hydrogel precursor experiences periodic shear cycling that enhances homogeneity of GAL distribution within the matrix prior to cold storage, and wherein the distilled water used for preparing the hydrogel precursor is characterized by electrical conductivity not exceeding 2 μS/cm, and wherein the water is filtered prior to use through a 0.22 μm membrane filter to remove particulate contaminants that would otherwise act as nucleation points for localized HPMC aggregation or GAL precipitation during the 10-minute shear mixing stage.

11. The method of claim 1, wherein during the two-day cold-swelling period at 4° C., the sealed container is positioned such that the hydrogel precursor occupies between 40% and 70% of the container volume, thereby creating a defined headspace above the hydrogel mixture, the headspace functioning to stabilize hydrogel development by enabling pressure equilibration while preventing any compressive resistance that might alter the hydration patterns of the HPMC molecular chains during the slow-temperature swelling progression, and wherein the sealed vessel is placed upon a vibration-dampening platform during the two-day storage period, the platform comprising a viscoelastic composite capable of attenuating oscillatory energy above 5 Hz, thereby ensuring that the hydrogel precursor remains undisturbed in a static spatial configuration essential for the uniform inward diffusion of water molecules into the polymer microdomains of the HPMC matrix.

12. The method of claim 1, wherein the final hydrogel mixture is subjected to visual inspection using a diffuse white-light source of 5600 K color temperature immediately following removal from cold storage, the inspection being performed while the parafilm seal remains intact, such that the operator evaluates internal phase uniformity through optical transmission and scattering characteristics without exposing the hydrogel matrix to ambient humidity exchange, and wherein the environmental conditions during the entire preparation procedure are controlled such that ambient relative humidity remains between 30% and 55%, and wherein no airflow exceeding 0.15 m/s is permitted across the open container during polymer hydration and GAL mixing steps, thereby preventing premature surface skin formation on the hydrogel precursor and ensuring consistent solvent evaporation rates during the active mixing interval.

13. The method of claim 1, wherein the hydrogel precursor is mixed within a vessel comprising cylindrical walls having a diameter-to-height ratio between 1:1 and 1:1.6, the vessel geometry being chosen to generate a stable, centrally aligned vortex during shear mixing, thereby ensuring consistent distribution of kinetic energy across the depth of the solution, uniform dissolution of GAL throughout the polymer matrix, and symmetric hydration of HPMC chains prior to the cold-swelling phase, and wherein the gradual incorporation of Galangin (GAL) into the HPMC dispersion further comprises maintaining the centerline temperature of the dispersion within ±0.5° C. of ambient temperature by periodically monitoring the thermal profile with a Class A glass laboratory thermometer, and wherein the temperature stabilization is performed to prevent thermally induced viscosity fluctuations within the polymeric medium, such that the GAL molecules experience a consistent microviscosity environment during solvation and interpenetration into the hydrating HPMC chain network throughout the entire 10-minute mixing interval.

14. The method of claim 1, wherein the hydrogel precursor is mixed in a vessel that has undergone a pre-conditioning step comprising rinsing with 70% ethanol, drying under laminar airflow, and subsequently equilibrating the vessel for at least 10 minutes to eliminate volatile residues, and wherein said pre-conditioning step is performed to ensure that the internal surface chemistry of the vessel remains inert with respect to GAL adsorption or polymer-surface interactions that may otherwise disrupt uniform matrix formation during HPMC hydration.

15. The method of claim 1, wherein the stirring operation is carried out at a rotational speed selected such that the vortex depth remains between 20% and 35% of the liquid column height, the vortex geometry enabling partitioning of the fluid into a high-shear central zone and a low-shear peripheral zone, and wherein GAL particles introduced into the vortex are entrained into the high-shear region for rapid dissolution before being advected toward the low-shear region, thereby producing cyclical solute redistribution within the hydrating HPMC matrix during mixing, and wherein the two-day cold storage at 4° C. is conducted inside a refrigeration system equipped with forced-air circulation restricted to an airflow velocity below 0.05 m/s at the shelf level, and wherein the hydrogel container is positioned at least 5 cm away from the refrigeration air outlets, thereby ensuring uniform cooling of the hydrogel precursor and preventing localized convective cooling patterns from influencing the polymer chain hydration symmetry across the entire bulk volume.

16. The method of claim 1, wherein the container sealed with parafilm is placed in an upright orientation during the cold-swelling phase, the upright positioning ensuring a gravitationally stable hydrogel-air interface, and wherein the hydrogel precursor is allowed to undergo unidirectional vertical hydration expansion from bottom to top, such that hydration gradients remain minimal due to the absence of lateral fluid displacement forces that would otherwise arise if the container were tilted or horizontally oriented during the swelling period.
